SEQUENCE LISTING (16 sequences)

SEQ ID NO: 1 - protein, 17 aa, Homo sapiens
  MOD_RES (1)..(1): PYRROLIDONE CARBOXYLIC ACID
  MOD_RES (17)..(17): AMIDATION
EGPWLEEEEEAYGWMDF

SEQ ID NO: 2 - protein, 18 aa, Homo sapiens
  MOD_RES (1)..(1): PYRROLIDONE CARBOXYLIC ACID
EGPWLEEEEEAYGWMDFG

SEQ ID NO: 3 - protein, 34 aa, Homo sapiens
  MOD_RES (1)..(1): PYRROLIDONE CARBOXYLIC ACID
  MOD_RES (34)..(34): AMIDATION
ELGPQGPPHLVADPSKKEGPWLEEEEEAYGWMDF

SEQ ID NO: 4 - protein, 35 aa, Homo sapiens
  MOD_RES (1)..(1): AMIDATION
ELGPQGPPHLVADPSKKEGPWLEEEEEAYGWMDFG

SEQ ID NO: 5 - protein, 6 aa, Homo sapiens
  MOD_RES (1)..(1): PYRROLIDONE CARBOXYLIC ACID
EGPWLE

SEQ ID NO: 6 - protein, 9 aa, Homo sapiens
  MOD_RES (9)..(9): AMIDATION
EEAYGWMDF

SEQ ID NO: 7 - protein, 6 aa, Homo sapiens
  MOD_RES (1)..(1): PYRROLIDONE CARBOXYLIC ACID
ELGPQG

SEQ ID NO: 8 - protein, 7 aa, Homo sapiens
YGWMDFG

SEQ ID NO: 9 - protein, 20 aa, Artificial sequence
  Lys-Lys-linked G17-Gly
KKEGPWLEEEEEAYGWMDFG

SEQ ID NO: 10 - protein, 12 aa, Artificial sequence
  Spacer copupled to G17 C terminus
  MOD_RES (12)..(12): AMIDATION
CSSEEAYGWMDF

SEQ ID NO: 11 - protein, 9 aa, Homo sapiens
EGPWLEEEE

SEQ ID NO: 12 - protein, 12 aa, Artificial Sequence
  G34 N terminal peptide coupled to spacer
  MOD_RES (1)..(1): PYRROLIDONE CARBOXYLIC ACID
ELGPQGRPPPPC

SEQ ID NO: 13 - protein, 8 aa, Homo sapiens [sequence omitted]

SEQ ID NO: 14 - protein, 14 aa, Artificial sequence
  Spacer coupled to C terminal peptide of G17-Gly
CPPPPSSYGWMDFG

SEQ ID NO: 15 - protein, 24 aa, Artificial sequence
  Spacer coupled to G34-Gly peptide
CGGSKKEGPWLEEEEEAYGWMDFG

SEQ ID NO: 16 - protein, 14 aa, Artificial sequence
  G17 N terminal peptide plus spacer
  MOD_RES (1)..(1): PYRROLIDONE CARBOXYLIC ACID
EGPWLEEEEAAPPC

(19) United States
(12) Patent Application Publication
(10) Pub. No.: US 2007/0066809 A1
(43) Pub. Date: Mar. 22, 2007

(54) Monoclonal antibodies to gastrin hormone

(76) Inventors: Stephen Grimes, Davis, CA (US); Ronald Makishima, North Highlands, CA (US)

Correspondence Address: FISH & RICHARDSON, PC, P.O. BOX 1022, MINNEAPOLIS, MN, 55440-1022, US

(21) Appl. No.: 11/499,261

(22) Filed: Aug. 3, 2006

Related U.S. Application Data
(63) Continuation of application No. 11/093,724, filed on Mar. 29, 2005, now abandoned.
(63) Continuation-in-part of application No. 10/813,336, filed on Mar. 29, 2004.
(60) Provisional application No. 60/557,759, filed on Mar. 29, 2004.
(60) Provisional application No. 60/458,244, filed on Mar. 28, 2003.

(51) Int. Cl. C07K 16/00

(52) U.S. Cl. 530/388.1

(57) ABSTRACT

The present invention provides monoclonal antibodies (MAbs) selective for the N-termini and C-termini of the gastrin hormone forms, gastrin-17 (G17), glycine-extended gastrin-17 (G17-Gly), gastrin-34 (G34) and glycine-extended gastrin-34 (G34-Gly); and the hybridomas that produce these MAbs. Also provided are panels of MAbs useful for the detection and quantitation of gastrin-17 (G17), glycine-extended gastrin-17 (G17-Gly), gastrin-34 (G34) and glycine-extended gastrin-34 (G34-Gly). These assays are useful for monitoring a gastrin-mediated disease or condition, or for monitoring the progress of a course of therapy. The invention further provides solid phase assays including immunohistochemical (IHC) and immunofluorescence (IF) assays suitable for detection and visualization of gastrin species in solid samples, such as biopsy samples or tissue slices. Pharmaceutical compositions of the MAbs of the invention are also provided, along with methods of diagnosis, prevention and treatment of gastrin-mediated diseases or conditions. Methods of evaluating a gastrin hormone-blocking treatment are described. The course of a gastrin-mediated disease or condition may be monitored in a patient by means of assay methods provided.

### RELATED APPLICATIONS

This application is co-filed on Mar. 29, 2004 with U.S. Ser. No. 10/______ entitled “Gastrin Hormone Immunoassays,” the specification of which is hereby incorporated by reference in its entirety.

### FIELD OF THE INVENTION

The invention relates to antibodies directed against specific regions of gastrin hormone and to the different forms of gastrin hormone found in vivo in an animal, particularly a human. The invention further relates to the application of these monoclonal antibodies (MAbs) to detection and diagnosis and monitoring of gastrin-mediated diseases and conditions, and to methods of use of the MAbs of the invention for the prevention and treatment of gastrin-mediated diseases and conditions.

### BACKGROUND OF THE INVENTION

Although gastrin hormone was first identified one hundred years ago, and was purified in the 1960's, its effects on different tissues in normal and disease tissues is still incompletely understood. One major reason for this gap in knowledge of the gastrin system has been the difficulty in separately detecting and quantifying each of the several forms of gastrin hormone.

In mammals the peptide hormone, gastrin exists in several forms, grouped into two major size classes, “little” gastrin and “big” gastrin, on the basis of the number of amino acid residues in the peptide chain. The “little” gastrin form includes mature gastrin-17 (G17) and glycine-extended G17 (G17-Gly); and “big” gastrin includes gastrin-34 (G34) and glycine-extended G34 (G34-Gly). The mature form of G17 is a major effector of stomach acid secretion and is estimated to be about six times more effective in this role than is G34. The various forms of gastrin are produced in vivo from a precursor peptide, progastrin by cleavage and in some cases, modification of the cleaved form. Human G34 has the entire seventeen amino acid sequence of G17 at its C-terminal end, and, predictably, cross-reacts immunologically with G17.

Mature G17 is modified at both amino- and carboxy-terminal residues: the N-terminal glutamic acid is cyclized to form pyroglutamic acid (pGlu) and the free carboxyl group of the C-terminal phenylalanine residue is amidated by the enzyme, peptidyl α-amidating mono-oxygenase (PAM) to form a C-terminal Phe-NH₂. Mature G34 is identically amidated at its C-terminal end to form a C-terminal Phe-NH₂. (See Dockray et al., Ann. Rev. Physiol. (2001) 63: 119-139).

Mature G17, the predominant form of “little” gastrin in humans, has the amino acid sequence: pEGPWLEEEEEAYGWMDF-NH₂ (SEQ ID NO: 1). G17-Gly is an incompletely processed form of gastrin found as a minor component of “little” gastrin in healthy human subjects and has the amino acid sequence: pEGPWLEEEEEAYGWMDFG (SEQ ID NO: 2).

Gastrin-34, the predominant form of “big” gastrin in humans, has the amino acid sequence: pELGPQGPPHLVADPSKKEGPWLEEEEEAYGWMDF-NH₂ (SEQ ID NO: 3), and glycine-extended gastrin 34 (G34-Gly), has an extra C-terminal glycine residue, having the amino acid sequence: pELGPQGPPHLVADPSKKEGPWLEEEEEAYGWMDFG (SEQ ID NO: 4).

Gastrin is secreted by the pyloric antral-G cells of the stomach in response to gastrin-releasing peptide (GRP), and is suppressed by gastric acid and the paracrine action of several peptide hormones, most notably, somatostatin. It has long been recognized that gastrin peptides function to stimulate acid secretion in the stomach of healthy individuals, however, it has only recently been shown that these peptides also control proliferation, differentiation and maturation of different cell types in the gastrointestinal (GI) system.

In addition to their local activity in the GI system, G17 and, to a lesser extent, G17-Gly are released into the bloodstream and have been found to increase in the serum of patients afflicted with gastrointestinal disorders and diseases, such as gastric cancer, colorectal cancer, and pancreatic cancer. These gastrin species have more recently also been found to be associated with other diseases not associated with the gastrointestinal tract, including small cell lung cancer (SCLC) and liver metastasized tumors. See for example “Gastrin and Colon Cancer: a unifying hypothesis” S. N. Joshi et al., Digestive Diseases (1996) 14: 334-344; and “Gastrin and Colorectal Cancer” Smith, A. M. and Watson, S. A. Alimentary Pharmacology and Therapeutics (2000) 14(10): 1231-1247.

Antibodies are key reagents in numerous assay techniques used in medical, veterinary and other fields. Such tests include many routinely used immunoassay techniques, such as for example, enzyme-linked immunosorbant assays (ELISA), radioimmunoassays (RIA), immunohistochemistry (IHC), and immunofluorescence (IF) assays.

Monoclonal antibodies (MAbs) have unique characteristics that render them superior in many respects to polyclonal antisera and to antibodies purified from polyclonal antisera when used in many of these assays. These attributes include monodeterminant specificity for the target antigen (i.e. specificity for a single epitope), unchanging specificity among different antibody preparations, as well as unchanging affinity and chemical composition over time. Furthermore, MAbs can be produced indefinitely and in unlimited amounts by in vitro methods. These properties are in sharp contrast to those of polyclonal antibodies, which require in vivo immunization methods with the unavoidable associated biological variability and limited antibody production capacity over the lifespan of the immunized animal.

Despite these advantages, differences exist between individual MAbs even though they may be specific for the same epitope. For example, differences between MAbs induced by immunization with a single antigenic epitope region can arise with respect to any or all of the following characteristics: 1) the fine specificity for the molecular composition and tertiary structure of the epitope; 2) the antibody idiotype; 3) the antibody affinity; 4) the antibody allotype; and 5) the antibody isotype. These characteristic differences can affect the behavior of MAbs in a particular immunoassay, such that different MAb isolates raised against the same antigenic region can behave differently in a given assay. Consequently, some MAbs will be superior to others that bind the same epitope when used as reagents in a particular immunoassay.

The immunoassay may be an enzyme-linked immunosorbent assay (ELISA), a radioimmunoassay (RIA), an immunodiffusion assay, or an immuno-detection assay, such as an ELISPOT, slot-blot, or a western blot. As a general guide to such techniques, see for instance, Ausubel et al. (eds) (1987) in “Current Protocols in Molecular Biology” John Wiley and Sons, New York, N.Y. Alternatively, the immunoassay may be an immunohistochemical (IHC) staining or immunofluorescence (IF) procedure for visualization of a form of gastrin hormone in a tissue sample. See for example “Principles and Practice of Immunoassay” (1991) Christopher P. Price and David J. Neoman (eds), Stockton Press, New York, N.Y.

Monoclonal antibodies selective for the N-terminal region and the C-terminal region of G17 have been described. See for example, Azuma et al., Gastroenterologica Japonica (1986) 21(4): 319-324; Ohning et al., Peptides (1994) 15(3):417-423; Fuerle et al., Pancreas (1995) 10(3):281-286; Kovacs et al., Peptides (1996) 17(4): 583-587; Ohning et al., Am. J. Physiol. (1996) 271(3 Pt 1):G470-476; Sipponen et al., (2002) Scand. J. Gastroenterol. 37(7): 785-791. However, none of these antibodies were shown, either alone or in combination, to be capable of distinguishing and quantifying more than one of the several forms of gastrin hormone found in biological fluids in normal and disease states.

Anti-gastrin polyclonal antibodies have been shown to be effective in inhibiting gastrin activity (“Inhibition of gastrin activity by incubation with antibodies to the C-terminal tetrapeptide of gastrin” Jaffe et al., Surgery (1969) 65(4):633-639); and non-human anti-gastrin polyclonal antibodies have been applied to therapy in a patient suffering from Zollinger-Ellison syndrome, a pathological condition in which excessive gastrin is produced without stimulation by feeding. See Hughes et al., “Therapy with Gastrin Antibody in the Zollinger-Ellison Syndrome” Hughes et al., Digestive Diseases (1976) 21(3):201-204. However, these rabbit anti-gastrin antibodies had “at best a short term effect in this patient.” (Hughes at p. 204). U.S. Pat. Nos. 5,886,128 and 5,785,970 disclose methods of treatment of ulcers or tumors whose growth is dependent on or stimulated by gastrin hormones by immunizing with gastrin hormone peptide conjugates.

Recently, the ratio of amidated to non-amidated forms of gastrin hormone in serum has been suggested as providing an indication of an individual's risk profile for developing duodenal ulcer disease or gastric atrophy. See published U.S. patent application 2003/0049689 entitled “Diagnosis and Treatment of Gastrointestinal Disease” of T. C. Wang. Another group has used a method that includes measuring fasting G17 levels as basis of an assessment of risk of gastric acid related disease by comparison with “cut-off values” of fasting G17 along with levels of pepsinogen I/II and an H. pylori marker. See WO 0423148 published Mar. 18, 2004.

Until now, MAbs capable of sensitively detecting, and accurately distinguishing each of the G17, G17-Gly, G34, and G34-Gly forms of gastrin hormone have not been available. Furthermore, until the present invention, it was not possible to accurately measure the amounts of each of these forms of gastrin hormone in a sample of biological fluid. Use of the MAbs of the invention in assays for clinical testing more precisely defines the biology of gastrin hormones in normal and disease states. Use of MAbs of the invention also provides MAb compositions for pharmaceutical use and methods for the prevention and treatment of gastrin-associated diseases and conditions.

### SUMMARY OF THE INVENTION

The present invention provides monoclonal antibodies (MAbs) that selectively bind the N-terminus of gastrin-17 (G17) or glycine-extended G17 (G17-Gly) at an epitope within the amino acid sequence pEGPWLE (corresponding to amino acids 1-6 of G17, SEQ ID NO: 5). Hybridomas that produce these MAbs that selectively bind the N-terminus of gastrin-17 (G17) or G17-Gly at an epitope within the amino acid sequence pEGPWLE (SEQ ID NO: 5) are also provided.

The present invention also provides MAbs that selectively bind the C-terminus of gastrin-17 (G17) or gastrin-34 (G34) at an epitope within the amino acid sequence EEAYGWMDF-NH₂ (SEQ ID NO: 6). Hybridomas that produce these MAbs that selectively bind the C-terminus of gastrin-17 (G17) or gastrin-34 (G34) at an epitope within the amino acid sequence EEAYGWMDF-NH₂ (SEQ ID NO: 6) are also provided.

The present invention further provides MAbs that selectively bind the N-terminus of human gastrin-34 (G34) at an epitope within the amino acid sequence of pELGPQG (SEQ ID NO: 7). Hybridomas that produce these MAbs that selectively bind the N-terminus of human gastrin-34 (G34) at an epitope within the amino acid sequence of pELGPQG (SEQ IID NO: 7) are also provided.

The present invention yet further provides MAbs that selectively bind the C-terminus of glycine-extended gastrin-17 (G17-Gly) and glycine-extended gastrin-34 (G34-Gly) at an epitope within the amino acid sequence of YGWMDFG (SEQ ID NO: 8). Hybridomas that produce these MAbs that selectively bind the C-terminus of glycine-extended gastrin-17 (G17-Gly) and glycine-extended gastrin-34 (G34-Gly) at an epitope within the amino acid sequence of YGWMDFG (SEQ ID NO: 8) are also provided.

Combinations of two or more of the antibodies of the invention can be used in a panel of MAbs that selectively bind the N-terminus or the C-terminus of each of the G17, G17-Gly, G34, and G34-Gly forms of gastrin hormone. Also provided are pharmaceutical compositions of a MAb that selectively binds: (1) the N-terminus of gastrin-17 (G17) or glycine-extended G17 (G17-Gly) at an epitope within the amino acid sequence pEGPWLE (corresponding to amino acids 1-6 of G17, SEQ ID NO: 5); (2) the C-terminus of gastrin-17 (G17) or gastrin-34 (G34) at an epitope within the amino acid sequence EEAYGWMDF-NH₂ (SEQ ID NO: 6); (3) the N-terminus of human gastrin-34 (G34) at an epitope within the amino acid sequence of pELGPQG (SEQ ID NO: 7); or (4) the C-terminus of glycine-extended gastrin-17 (G17-Gly) and glycine-extended gastrin-34 (G34-Gly) at an epitope within the amino acid sequence of YGWMDFG (SEQ ID NO: 8); in combination with a pharmaceutically acceptable carrier.

Gastrin-mediated diseases or conditions in a patient can be diagnosed by determining the level of a form of gastrin hormone in a sample of a biological fluid from the patient and comparing the level of a form of gastrin hormone in the sample with the normal level of the gastrin hormone form in a sample of biological fluid from a group of healthy individuals.

Such gastrin-mediated diseases or conditions can be prevented or treated by administering to a patient in need thereof a pharmaceutical composition including a MAb that selectively binds: (1) the N-terminus of gastrin-17 (G17) or glycine-extended G17 (G17-Gly) at an epitope within the amino acid sequence pEGPWLE (corresponding to amino acids 1-6 of G17, SEQ ID NO: 5); (2) the C-terminus of gastrin-17 (G17) or gastrin-34 (G34) at an epitope within the amino acid sequence EEAYGWMDF-NH₂ (SEQ ID NO: 6); (3) the N-terminus of human gastrin-34 (G34) at an epitope within the amino acid sequence of pELGPQG (SEQ ID NO: 7); or (4) the C-terminus of glycine-extended gastrin-17 (G17-Gly) and glycine-extended gastrin-34 (G34-Gly) at an epitope within the amino acid sequence of YGWMDFG (SEQ ID NO: 8).

A method of monitoring the course of a gastrin-mediated disease or condition in a patient is also provided. The method includes determining the level of a gastrin hormone form in a sample of a biological fluid from a patient suffering from or at risk of a gastrin-mediated disease or condition at a first time point; determining the level of the gastrin hormone form in one or more samples of the biological fluid from the patient at different time points; and thereby monitoring the course of the gastrin-mediated disease or condition.

The invention also provides a method of evaluating a gastrin hormone-blocking treatment of a patient suffering from a gastrin hormone-mediated disease or condition. The method includes the following steps (a)-(j):
a) obtaining a first sample of biological fluid from the patient prior to or in the early stages of a treatment;
b) determining the level of gastrin hormone in the first sample by an immunoassay method;
c) performing a diagnosis on the basis of the disease or condition to be treated and the level of gastrin hormone in the first sample;
d) administering a treatment to the patient, comprising: a first agent or a substance that generates a first agent which binds gastrin hormone so as to modulate its binding to its target receptor in vivo;
e) obtaining a second sample of biological fluid from the patient after a suitable time within which the treatment would have an effect;
f) determining the level of total gastrin hormone including bound and free gastrin hormone in a first aliquot of the second sample by an immunoassay method, wherein the first aliquot of the second sample is incubated with (i) a second agent that displaces any gastrin hormone bound by the first agent, and (ii) an immobilized anti-gastrin hormone antibody, wherein the immobilized antibody does not bind the second agent; washing to remove the second agent and adding a detectable antibody that binds the gastrin hormone and does not compete with the immobilized antibody, forming an immunocomplex comprising the immobilized antibody bound to gastrin hormone, the gastrin hormone in turn being bound by the detectable antibody;
g) detecting the amount of the detectable antibody in the immunocomplex and thereby determining the amount of total gastrin hormone in the second sample;
h) determining the level of free gastrin hormone by repeating steps f) and g) with a second aliquot of the second sample, wherein the incubation in step f) is performed without the second agent; and
j) comparing the determined amounts of free gastrin hormone in the first sample with the amounts of free and total gastrin hormone in the second sample so as to determine the efficacy of the gastrin hormone-blocking treatment in the patient.

The invention further provides a kit for performing an immunoassay including an anti-gastrin hormone MAb and a suitable container. Preferably the anti-gastrin MAb is selected from the group consisting of the following MAbs: 400-1, 400-2, 400-3, 400-4, 401-2, 445-1, 445-2, and 458-1.

### BRIEF DESCRIPTION OF THE FIGURES

FIG. 1: ELISA with hG17-BSA coated plates. A plot of Absorbance at 405 nm (A₄₀₅) against titer of the following sera: Squares represent the test sample. Diamonds represent the pre-bleed. Triangles represent the reference standard. The Absorbance (2) obtained at a 2×10⁵ titer (1) of the positive standard is determined. The point at which the Test Sample's curve bisects this absorbance indicates the titer of the Test Sample (3). In this Example, the Test Sample has a titer of 2.8×10⁴.

FIG. 2. A representative calibration curve for total gastrin-17 showing gastrin concentration in picomoles plotted against absorbance at 450 nm (A₄₅₀) to follow the enzymatic development using tetramethylbenzidine sulfonate (TMBS) chromogen.

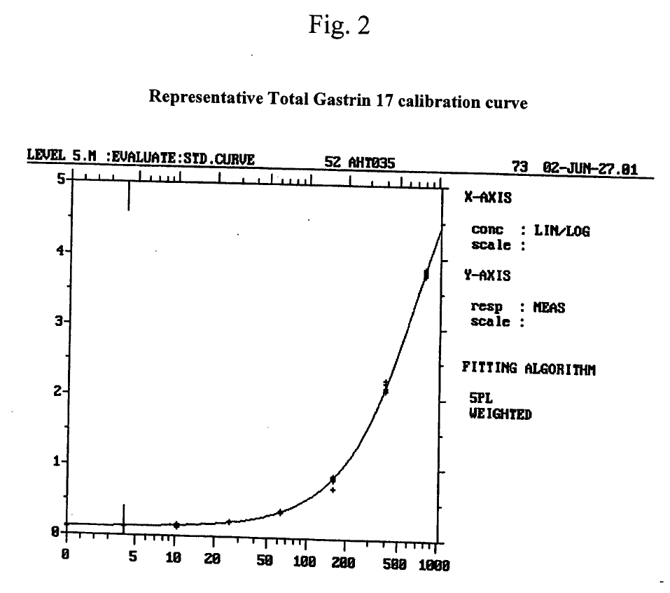

FIG. 3. A representative calibration curve for free gastrin-17 showing gastrin concentration in picomoles plotted against absorbance at 450 nm (A₄₅₀) as described above.

### DETAILED DESCRIPTION OF THE INVENTION

The following provides the definitions of terms and phrases as used in this specification:

A “gastrin hormone” or “gastrin hormone form” as used interchangeably herein means any biologically active and/or immunologically cross-reactive gastrin hormone peptide. The major forms of gastrin hormone include, but are not limited to gastrin-17 (G17), whether amidated at the C-terminus or having a free C-terminus; glycine extended gastrin-17 (G17-Gly); gastrin-34, (G34) including both the C-terminally amidated form and the form having a free C-terminus; glycine extended gastrin-34 (G34-Gly), and progastrin.

The “total amount” of a gastrin hormone form in a sample as used herein means the sum of the amount of free (unbound) gastrin hormone form plus the amount of complexed (bound) gastrin hormone form. The complexed gastrin may be bound by an antibody or other binding moiety in the sample.

A “biological fluid” as used herein means any fluid that includes material of biological origin. Preferred biological fluids for use in the present invention include bodily fluids of an animal, especially a mammal, preferably a human subject. The bodily fluid may be any bodily fluid, including but not limited to blood, plasma, serum, lymph, cerebrospinal fluid (CSF), and the like.

A “preservative agent” as used herein means any agent, supplement or additive that reduces the time dependent degradation of gastrin in a sample of biological fluid, or a liquid sample comprising a biological component. Preservative agents useful in the practice of the present invention include any of the many preservative agents well known in the art, including but not limited to general chemical preservatives, such as for instance, sodium azide, EDTA and protease inhibitors, such as for instance, PMSF (Phenylmethylsulfonylfluoride), and aprotinin (e.g. Trasylol), or a biological preservative, such as for instance, heparin.

New Anti-Gastrin Monoclonal Antibodies

Selection of the optimal monoclonal antibody (MAb) for use in a particular application is preferably achieved by assessing the performance of each of the individual candidate MAbs in the end application. For this reason, testing of candidate MAbs for optimum functionality in the intended end application is part of the selective process to derive a MAb that is optimal for the intended use. This selective step is performed in addition to the selection steps normally undertaken in deriving MAbs, which include binding to the targeted antigen and serial cloning of the hybridoma that produces the MAb to ensure stability of the essential characteristics of the hybridoma cell line, including persistent cell growth and division, and consistent unlimited antibody production over an indefinite period.

As used herein, the term “selective” for a particular a form of gastrin hormone means that the antibody, while being specific for the particular target epitope of a particular form of gastrin hormone, binds each of the forms of gastrin hormone that contain the target epitope. For instance, the C-terminal of mature (amidated) G17 is common to mature G17 and G34. Thus, a MAb that is specific for the target C-terminal epitope found on mature G17 C-terminus is selective for G17 (and for G34).

Specifically, the present invention discloses a method of identifying MAbs selective for the N-terminal and C-terminal of the biologically active forms of gastrin hormone, amidated gastrin-17 (G17), amidated gastrin-34 (G34), glycine-extended gastrin 17 (G17-Gly), glycine-extended gastrin 34 (G34 Gly), and progastrin wherein the MAbs have superior properties. These MAbs are particularly suitable for use in an immunoenzymometric assay (commonly termed an “ELISA” or enzyme-linked immunosorbent assay) designed to measure the particular form of gastrin hormone in a biological fluid. The MAbs of the present invention are also suitable for detecting and/or quantifying gastrin hormone in immunodetection assays, such as for instance ELISPOT, radioimmunoassay, antibody-based sandwich capture assays, dot-blot, slot blot and western blot assays.

In one aspect, the present invention provides MAbs that selectively bind the N-terminus of gastrin-17 (G17) at an epitope within the amino acid sequence pEGPWLE (SEQ ID NO: 5). The binding of these MAbs selective for the N-terminus of gastrin-17 (G17) to the BSA-conjugate of peptide pEGPWLEEEE (SEQ ID NO: 11) is inhibited by human G17, equine G17 or human G17-Gly.

In another aspect, the present invention provides MAbs that selectively bind the C-terminus of gastrin-17 (G17) or gastrin-34 (G34) at an epitope within the amino acid sequence EEAYGWMDF-NH₂ (SEQ ID NO: 6).

In a further aspect, the present invention provides MAbs that selectively bind the N-terminus of human gastrin-34 (hG34) at an epitope within the amino acid sequence pELGPQG (SEQ ID NO: 7).

In yet another aspect, the present invention provides MAbs that selectively bind the C-terminus of glycine-extended gastrin-17 (G17-Gly) and glycine-extended gastrin-34 (G34-Gly) at an epitope within the amino acid sequence YGWMDFG (SEQ ID NO: 8).

In yet a further aspect, the invention provides MAbs that selectively bind progastrin. These Mabs bind progastrin, but do not bind the processed gastrin hormone forms: G17, G34, G17-Gly or G34-Gly. The MAbs of the invention selective for progastrin include MAbs that bind the C-terminus of human progastrin. These MAbs will also bind preprogastrin, which consists of a peptide chain of 101 amino acids from which progastrin, and gastrin are sequentially processed. However, processing of preprogastrin is rapid and occurs at the endoplasmic reticulum (ER) where it is synthesized. The MAbs of the invention that bind progastrin are useful in assays described herein to detect and quantitate progastrin in a sample.

The MAbs of the invention preferably bind the gastrin form for which they exhibit selective binding with an association constant (Kₐ) of from about 10⁶ to about 10⁷ LM⁻¹, preferably from about 10⁷ to about 10⁸ LM⁻¹, yet more preferably from about 10⁸ to about 10⁹ LM⁻¹, even more preferably from about 10⁹ to about 10¹⁰ LM⁻¹, and still more preferably from about 10¹⁰ to about 10¹¹ LM⁻¹, and most preferably from about 10¹¹ to about 10¹² LM⁻¹.

Panels of Anti-Gastrin Monoclonals

The present invention provides for the first time panels of anti-gastrin hormone MAbs that permit unequivocal identification and quantitation of more than one of the G17, G17-Gly, G34, and G34-Gly forms of gastrin hormone. For example, a panel of MAbs that includes a MAb selective for the N-terminus of the G34 form of gastrin hormone and a MAb selective for the C-terminus of G17/G34 (the C-terminus of G34 is identical to the C-terminus of G17) allows the specific identification and quantitation of G34 in a sample by any one of a number of immunoassays that are routine in the art. Routine immunoassays in which the MAbs of the invention may be used include, but are not limited to, enzyme-linked immunosorbent assays (ELISAs), radioimmunoassays (RIAs), immunofluorescence assays (IFs), immunohistochemical assays (IHCs), immunodiffusion assays and the like. See for instance U.S. Pat. No. 5,932,412 entitled “Synthetic peptides in human papilloma virus 1, 5, 6, 8, 11, 16, 18, 31, 33 and 56 useful in immunoassay for diagnostic purposes” to Dillner et al. for examples of such routine diagnostic assay methods.

Supplementation of the panel of MAbs with one or more additional MAbs of the invention provides the capability of specific identification and quantitation of further gastrin hormone species in a sample. For example, addition of a MAb selective for the N-terminus of the G17 form to the above-described panel of antibodies further permits the specific identification and quantitation of free and total (bound plus free) G17 hormone in a sample by methods of the present invention as described below.

Similarly, a panel of MAbs that includes a MAb selective for the N-terminus of the G34 and a MAb selective for the C-terminus of glycine-extended G34 (which is identical to the C-terminus of glycine-extended G17) allows the specific identification and quantitation of glycine-extended G34. in a sample. Furthermore, addition to the panel of a MAb selective for the N-terminus of G17 permits the identification and quantitation of free and total (bound plus free) glycine-extended G17 in the sample as described herein.

Other combinations of pairs of MAbs selected from the MAbs of the invention, useful in a panel of MAbs for identification, quantitation and monitoring of other forms of gastrin hormone will be immediately apparent to those of skill in the art. The present invention encompasses all such pairs of MAbs of the invention and combinations of pairs of MAbs of the invention and any other groupings of pairs of MAbs of the invention.

The MAbs of the present invention provide the means to accurately determine the amounts and ratios of gastrin hormone forms for assessment of predispositions to gastrin-hormone-mediated diseases and conditions, and for detection and diagnosis of such diseases and conditions in patients suffering therefrom. For example, the anti-gastrin MAbs of the invention can be incorporated into ELISA assays for large scale screening of patient serum or other biological fluid, for any one or all of the G17, G34, and the G17-Gly, and G34-Gly gastrin hormone forms.

The MAbs of the present invention, combinations of pairs of MAbs selected from the MAbs of the invention, and panels of MAbs of the present invention are particularly useful when applied to high-throughput methods. Such methods include micro-chip and micro-array methods of gastrin hormone antigen detection, such that many samples can be tested on a microplate or slide, or other assay substrate, such as a plate with virtual wells (such as for instance, that described in U.S. Pat. No. 6,565,813 to Garyantes et al). Detection of binding can be by any one of the many state-of-the-art detection systems currently available. Detection of binding can be, for instance, by surface plasmon resistance changes caused by specific biomolecular reactions, such as antigen-antibody binding. See for example, U.S. Pat. No. 5,981,167 to Taremi et al. for an application of this technology to enzymatic assays. The technology may be applied in a continuous flow mode and is equally applicable to detection of antibody binding to a surface-immobilized peptide or protein, such as a gastrin hormone, or to the detection of a gastrin-antibody complex. The latter complex may be detected by binding to a surface immobilized antibody specific for an epitope of the form of gastrin hormone (G17, G34, G17-Gly or G34-Gly) that is not sterically hindered by the antibody of the complex. Furthermore, this technology has the advantage of high throughput applicability and high sensitivity without the requirement for a radiolabel.

The MAbs of the present invention are also useful for immunohistochemical (IHC) and immunofluorescence (IF) assays of tissue samples, such as for instance, from biopsy material. Such analyses can be used to detect aberrant levels of individual gastrin-hormone forms and hence to diagnose gastrin-hormone-mediated diseases and conditions.

The MAbs of the present invention can be humanized according to established techniques well known in the art. See for instance, U.S. Pat. No. 6,689,869 entitled “Labeled humanized anti-CD-18 antibodies and fragments and kits” to Waldman et al., and U.S. Pat. No. 6,639,055 entitled Method for making humanized antibodies” to Carter et al. The humanized antibody can be reshaped to more closely match the binding affinity of the original mouse Mab. See for instance, U.S. Pat. No. 6,699,974 entitled “Re-shaped human anti-HM1.24 antibody” to Ono et al.

The Mabs of the present invention are also usefull for prevention and therapy of gastrin-hormone-mediated diseases and conditions. The anti-gastrin MAbs of the invention can be formulated in pharmaceutical compositions for passive immunization against particular gastrin hormone forms. See for example, U.S. Pat. No. 6,391,299 (herein after the '299 patent) entitled “Anti-factor IX/IXa antibodies” to Blackburn et al. Functional fragments of the MAbs of the present invention, such as, for instance Fab fragments, F(ab′)₂ fragments and any fragments (see the '299 patent for fragment descriptions) that retain the ability to bind the gastrin hormone form to which they are directed can also be incorporated into pharmaceutical compositions and applied in therapy. See the '299 patent for useful pharmaceutical compositions. The preferred routes of administration of the pharmaceutical compositions of the invention include parenteral routes of administration, such as subcutaneously, intramuscularly and intravenously. Alternatively, the pharmaceutical compositions can be delivered intranasally. Such pharmaceutical compositions are particularly usefull when administered in an effective amount for the prevention or therapy of gastrin-hormone-mediated diseases or conditions in patients having a prognosis of high likelihood of such diseases or conditions, or for the treatment of patients already suffering from such diseases or conditions.

An effective amount of a pharmaceutical composition that includes an intact or functional fragment of an anti-gastrin MAb, particularly a humanized anti-gastrin MAb of the invention for the treatment of a gastrin-mediated disease or condition is defined as an amount that prevents onset of or reduces the rate of progression of the disease or condition: more preferably an effective amount is an amount that stabilizes the disease or condition; more preferably still an effective amount is an amount that causes regression of the disease or condition. Most preferably, an effective amount is an amount that completely cures the disease or condition.

Furthermore, the MAbs of the present invention can be applied in immunoassays for monitoring the progression of gastrin-hormone-mediated diseases and conditions, where the level or amount of particular gastrin hormone forms, or of free, or bound or total gastrin forms provides an indication of the success of treatment or therapy, or of progression of the gastrin-hormone-mediated disease or condition.

Moreover, the MAbs of the present invention are useful in methods of evaluating a gastrin hormone blocking treatment of a patient suffering from a gastrin hormone-mediated disease or condition. The method includes the steps of:

a) obtaining a first sample of biological fluid from the patient prior to or in the early stages of a treatment;

b) determining the level of gastrin hormone in the first sample by an immunoassay method;

c) performing a diagnosis on the basis of the disease or condition to be treated and the level of gastrin hormone in the first sample;

d) administering a treatment to the patient, comprising: a first agent or a substance that generates a first agent which binds gastrin hormone so as to modulate its binding to its target receptor in vivo;

e) obtaining a second sample of biological fluid from the patient after a suitable time within which the treatment would have an effect;

f) determining the level of total gastrin hormone including bound and free gastrin hormone in a first aliquot of the second sample by an immunassay method, wherein the first aliquot of the second sample is incubated with (i) a second agent that displaces any gastrin hormone bound by the first agent, and (ii) an immobilized anti-gastrin hormone antibody, wherein the immobilized antibody does not bind the second agent; washing to remove the second agent and adding a detectable antibody that binds the gastrin hormone and does not compete with the immobilized antibody, forming an immunocomplex comprising the immobilized antibody bound to gastrin hormone, the gastrin hormone in turn being bound by the detectable antibody;

g) detecting the amount of the detectable antibody in the immunocomplex and thereby determining the amount of total gastrin hormone in the second sample;

h) determining the level of free gastrin hormone by repeating steps f) and g) with a second aliquot of the second sample, wherein the incubation in step f) is performed without the second agent; and

j) comparing the determined amounts of free gastrin hormone in the first sample with the amounts of free and total gastrin hormone in the second sample so as to determine the efficacy of the gastrin blocking treatment in the patient.

The above-described method applied to evaluating a gastrin hormone-blocking treatment in a patient is particularly valuable in clinical practice, where timing of decisions to proceed with one therapeutic regimen or another may be critical to the outcome for the patient. The method of the present invention provides information on which to base these critical decisions. The method provides a measure of gastrin hormone prior to or in the early stages of treatment (e.g. shortly after immunization with a gastrin hormone peptide conjugate vaccine, such as that described in U.S. Pat. No. 5,622,702) and provides one or more measurements of total and/or free gastrin hormone after a period in which the treatment is expected to have begun to be effective.

The gastrin hormone-blocking treatment may be active immunization, wherein an immunogen that raises antibodies to gastrin is administered to the patient as mentioned above. Alternatively, a gastrin hormone-blocking substance may be passively administered to the patient. The gastrin hormone-blocking substance may any gastrin hormone-blocking substance, including but not limited to an anti-gastrin hormone antibody, particularly a humanized monoclonal anti-gastrin hormone antibody; or the gastrin hormone-blocking substance may be a gastrin hormone receptor or a gastrin hormone receptor-mimic. The gastrin hormone receptor-mimic may be any molecule that mimics gastrin hormone receptor binding to gastrin hormone, such as for instance, a soluble gastrin hormone receptor or soluble gastrin hormone receptor fragment, or any other molecule that is functional in binding gastrin hormone.

The present invention also provides compositions, methods and kits for screening samples suspected of containing gastrin hormone. Such screening may be performed on patient samples, or laboratory samples suspected of containing or producing such a polypeptide. A kit can contain an antibody of the present invention. The kit can contain reagents for detecting an interaction between a sample an antibody of the present invention. The provided reagent can be radio-, fluorescently- or enzymatically-labeled. The kit can contain a known radiolabeled agent capable of binding or interacting with an antibody of the present invention.

The reagent of the kit can be provided as a liquid solution, attached to a solid support or as a dried powder. When the reagent is provided in a liquid solution, preferably, the liquid solution is an aqueous solution. Preferably, when the reagent provided is attached to a solid support, the solid support can be chromatographic media, a test plate having a plurality of wells, or a microscope slide. When the reagent provided is a dry powder, the powder can be reconstituted by the addition of a suitable solvent, that may be provided.

The kit of the invention is provided in a container that generally includes a vial into which the antibody, antigen or detection reagent may be placed, and preferably suitably aliquotted. The kits of the present invention will also typically include a means for containing the antibody, antigen, and reagent containers for commercial sale. Such containers may include plastic containers into which the desired vials are retained and one or more necessary chemicals, such as chromatography material, solvents and eluents, test tubes, detergents, antibodies and chemicals for the detection reaction.

In still further embodiments, the present invention concerns immunodetection methods and associated kits. It is proposed that gastrin hormone or peptide fragments thereof may be employed to detect antibodies having reactivity therewith, or, alternatively, antibodies prepared in accordance with the present invention, may be employed to detect gastrin hormone or gastrin hormone-mediated epitope-containing peptides. In general, these methods will include first obtaining a sample suspected of containing such a hormone, peptide or antibody, contacting the sample with an antibody or peptide in accordance with the present invention, under conditions effective to allow the formation of an immunocomplex, and then detecting the presence of the immunocomplex.

In general, the detection of immunocomplex formation is quite well known in the art and may be achieved through the application of numerous approaches. For example, the present invention contemplates the application of ELISA, RIA, immunoblot (e.g., dot blot, slot blot, western blot etc.), indirect immunofluorescence techniques and the like. Generally, immunocomplex formation will be detected through the use of a label, such as a radiolabel or an enzyme tag (such as alkaline phosphatase, horseradish peroxidase, or the like). Additional advantages may accrue through the use of a secondary binding ligand such as a second antibody or a biotin/avidin ligand binding arrangement, according to methods well known in the art.

### EXAMPLE 1

### Production of Monoclonal Antibodies to the C-terminal of Human G17

The peptide, CSSEEAYGWMDF-NH₂ (SEQ ID NO: 10) containing the linker-spacer (-Cys-Ser-Ser-) sequence followed by the amino acid sequence including C-terminal epitopes of human G17 and G34 (-EEAYGWMDF-NH₂, SEQ ID NO: 6) was synthesized commercially by standard solid phase peptide synthesis methodology.

The peptide was incorporated into an immunogen to induce antibodies to the C-terminus of G17/G34 as follows: The peptide was first covalently linked to diphtheria toxoid (“DT”) to yield a peptide-carrier conjugate. The number of peptide units substituted on each DT carrier was determined and finally, the conjugate was formulated as an immunogen. The techniques used were as described in U.S. Pat. No. 5,622,702.

Briefly, the chemical conjugation of peptide to carrier was conducted with the heterobifunctional cross-linker, epsilon-maleimidocaproic acid N-hydroxysuccinimide (ε-MCS). The conjugate was purified by dialysis against 0.1M sodium phosphate buffered saline, pH 7.3 (PBS) and the protein concentration determined by the Lowry assay. The substitution level of peptide on DT was determined on a molar basis by amino acid analysis of the conjugate. The dissolved conjugate was then formulated as an immunogen with Montanide ISA 703 (SEPPIC, France) as adjuvant by mixing the conjugate solution with the Montanide ISA 703 oil at a 30/70 ratio (wt/wt of conjugate/adjuvant). Mixing was achieved by drawing the appropriate volumes of each liquid into a syringe and then rapidly passing the solutions back and forth between a second syringe through an inter-locking hub.

Mice were initially immunized by i.p. injection with 0.1 mg of the peptide-DT conjugate immunogen/Montanide ISA 703 in a volume of 0.1 mL. A second injection of an identical dose was given three weeks after the first injection.

To create hybridomas producing a MAb selective for the C-terminal of G17/G34, spleen cells from the immunized mice were fused with a standard mouse myeloma fusion partner cell line by standard techniques well known to those skilled in the art. These methods are described in many reviews and laboratory handbooks. See, for instance, U.S. Pat. No. 4,196,265 Method of producing antibodies to Kaprowski et al; “Selected Methods in Cellular Immunology” (Chapter 17: Immunoglobulin Producing Hybrid Cell Lines, B. Mishell and S. Shiigi, W.H. Freeman and Co., San Francisco, 1980); Harlowe and Lane, Antibodies: A Laboratory Manual, Cold Spring Harbor Laboratory Press, 1988; Zola, Monoclonal Antibodies: A Manual of Techniques, CRC Press, Inc., Boca Raton, Fla., 1987. Immunized mice were boosted with an i.p. injection of 0.1 mg of the above-described peptide-DT conjugate in PBS 4 days prior to collection of their spleen cells for the cell fusion. Initial selection of hybrid cells was done using hypoxanthine-aminopterin-thymidine supplemented media, as described in Mishell and Shiigi. This fusion was designated F458.

The first selection steps for isolating hybridomas producing MAbs to the C-terminal end of G17 comprised selection of cells for production of antibody to the target peptide and for stability of the hybrid cell lines. The selection of cells producing antibody was accomplished by screening cell culture media obtained from tissue culture wells containing single clones for antibody to the C-terminal end of G17/34. The screening was accomplished by means of an ELISA using as target antigen a conjugate comprising an amidated synthetic peptide (amino acids 16-34—NH₂ of G34), linked at lysine-16 through a cysteine to bovine serum albumin (BSA) as an immunological carrier. Suitable ELISA techniques are known to those skilled in the art, and several examples are specifically described below. Stable cell lines were obtained by twice cloning each hybrid that produced antibodies that bound the hG34₍₁₆₋₃₄₎NH₂-BSA conjugate in the ELISA test. By means of these methods, fifteen hybrid cell lines were obtained that produced MAbs to the C-terminal common to G17 and G34.

### EXAMPLE 2

### Selection of Monoclonal Antibodies with Superior Performance in an Immunoenzymometric Assay for Total (Bound Plus Free) G17

A method for measuring the total quantity of G17 in samples of a biological fluid, such as human plasma that may contain anti-gastrin antibodies has been developed and is described in co-filed patent application U.S. Ser. No. 10/______. Briefly, the method includes adding to a test sample of a biological fluid an excess amount of a peptide comprising amino acids 1-8 of human G17 (human G17₍₁₋₈₎ displacement peptide), to displace any gastrin hormone that may be present and bound through an N-terminal epitope to G17 N-terminal epitope specific antibodies that might also be present in the test sample. After an incubation period, the sample mixture containing the displacing peptide is added to a 96-well ELISA plated coated with capture antibody directed to the C-terminal of G17. Following incubation, the plate is washed to remove the displacing peptide, and bound G17 is subsequently detected and quantified by the addition of an enzyme-linked antibody that binds an N-terminal epitope of G17. Another series of washing steps are necessary to remove unbound enzyme-linked antibody, and detectable signal is developed by addition of a chromogenic or other substrate that produces a detectable product by the action of the enzyme linked to the antibody. For example, when the enzyme is horseradish peroxidase (HRP) the substrate is tetramethylbenzidine sulfonate (TMBS). When alkaline phosphatase is the enzyme used for detection, p-nitrophenolphosphate can be used as the chromogenic substrate producing the colored compound p-nitrophenol. The degree of color development, read as Absorbance Units (AU, read at 405 nm in the case of p-nitrophenol, or at 450 nm in the case of TNBS) is indicative of the amount of G17 present in the test sample, and the actual concentration is determined by reading absorbance of the test sample against a standard curve generated with known concentrations of G17.

Preliminary tests were run with this assay using plasma samples from human patients to which G17 was added to predetermined concentrations, and performing the immunoassay with polyclonal rabbit antibodies against the C-terminal epitope of G17 as capture antibody coated onto the wells of the test plate. The results and data obtained from these assays showed poor consistency and did not provide an acceptable level of sensitivity. Therefore, C-terminal selective MAbs were used to coat the test plates and tested as capture antibodies in the assay.

To test each of the C-terminal G17-selective MAbs from fusion 458 in the total G17 assay, the fifteen individual MAbs to the C-terminal end of G17 were first purified by protein G affinity chromatography. This was done using a commercial kit (HiTrap Protein G HP, 1 mL, Amersham Biosciences) according to the manufacturer's instructions. The concentration of each MAb was then determined from the absorbance at a wavelength of 280 nm (A₂₈₀). The A₂₈₀ was divided by the concentration coefficient of 1.4 mL/mg to give the concentration. The concentrations were adjusted to fall in the range of 0.1-1.0 mg/mL. Each solution was then re-tested (undiluted) in the ELISA to qualitatively confirm the binding of the MAb to the C-terminal end of G17.

The fifteen Mabs, one negative control MAb and 3 mixtures of the purified Mabs, were then tested for performance in the immunoenzymometric assay for total G17 using the human G17₍₁₋₈₎ displacement peptide as described above. Each of the MAbs was diluted to a final concentration of 10 ug/mL in assay coating buffer (One vial of Convol pH 8.0 concentrated buffer solution (BDH product 18052 1U) added to 2.5 L of water; sodium azide (2.5 g) added and dissolved), then used to coat wells of a 96-well ELISA plate (0.1 mL added per well), as described above in the method for measurement of total G17.

An aliquot of a known concentration of G17 was added to a serum sample that was depleted of natural G17 by incubating overnight at room temperature to allow the endogenous serum proteases to digest any G17 present. Dilutions of this “G17-spiked” serum were made to prepare standard solutions of known concentrations of G17. The concentrations of G17 in the standards were 0, 4.1, 64 and 800 pM. These samples were then treated as test samples in the assay, with the addition of human G17₍₁₋₈₎ displacement peptide comprising the N-terminal of G17. Each of the G17 solutions was then added to the plate wells coated with the individual G17 C-terminal selective MAb preparations, and the total G17 assay was run according to the procedure described above.

The results of these assays, shown in A₂₈₀ absorbance units obtained with each concentration of G17 in the assay using each of the fifteen monoclonal antibodies coated on the wells of the test plate as capture MAb, are given in Table 1.

TABLE 1
Test of Individual Monoclonal Antibodies against the C-terminal
of G17 for performance in an ELISA for total G17.

| | Coating antibody ID, mean response (AU) | | | | |
|---|---|---|---|---|---|
| Standard | F458-4 | F458-4 | | | |
| value | 10H 6A | 7E 1H | F458-3 | F458-4 | F458-2-5F |
| (pM) | 3G | 4D | 7G 7D 11B | 12A 4H 8C | 8A 1A |
| 0 | 0.016 | 0.092 | 0.097 | 0.143 | 0.022 |
| 4.1 | 0.014 | 0.146 | 0.131 | 0.215 | 0.028 |
| 64 | 0.015 | 0.399 | 0.318 | 0.719 | 0.063 |
| 800 | 0.017 | 2.705 | 2.678 | 3.596 | 0.484 |

| | Coating antibody ID, mean response (AU) | | | | |
|---|---|---|---|---|---|
| Standard | F458-2- | | | | |
| value | 11A | F458-1-1E | F458-3-8G | F458-1-8E | F458-4-7C |
| (pM) | 8D 8C | 7B | 1H 3C | 7C 5G | 9B 8B |
| 0 | 0.065 | 0.108 | 0.099 | 0.134 | 0.150 |
| 4.1 | 0.088 | 0.191 | 0.204 | 0.210 | 0.266 |
| 64 | 0.459 | 0.327 | 0.605 | 0.492 | 0.681 |
| 800 | 3.135 | 2.649 | 3.642 | 3.439 | 3.784 |

| | Coating antibody ID, mean response (AU) | | | | |
|---|---|---|---|---|---|
| Standard | F458-4- | | | | |
| value | 12G | F458-4-6E | F458-1-7A | F458-3-1G | F458-4-5E |
| (pM) | 7E 3E | 4C 4A | 3H 1D | 9C 12A | 4H 10A |
| 0 | 0.020 | 0.086 | 0.156 | 0.070 | 0.108 |
| 4.1 | 0.025 | 0.208 | 0.168 | 0.042 | 0.175 |
| 64 | 0.042 | 0.580 | 0.291 | 0.079 | 0.316 |
| 800 | 0.109 | 3.229 | 3.260 | 0.864 | 2.950 |

| | Coating antibody ID, mean response (AU) | | | |
|---|---|---|---|---|
| Standard | F458-2-11B | F458 | | |
| value (pM) | 7A 11H | Pool #1 | F458-Pool #2 | F458 Pool #3 |
| 0 | 0.023 | 0.129 | 0.294 | 0.211 |
| 4.1 | 0.028 | 0.212 | 0.162 | 0.249 |
| 64 | 0.067 | 0.423 | 0.382 | 0.640 |
| 800 | 0.415 | 3.027 | 2.999 | 3.799 |

The optimum MAb was selected on the basis of the results from testing the performance of each MAb in the assay. The criteria used for comparing the isolated MAbs included the following:
1) a low absorbance value for 0.0 pM G17 added (baseline value, preferable ≦0.1 AU);
2) absorbance of double the baseline at 4.1 pM G17;
3) steepest increase (slope) in AU between 4.1 pM and 64 pM G17, the major working range of the assay; and
4) highest in AU for the 800 pM concentrations of G17.

Based on these criteria, the MAb that performed best was F458-3-8G 1H 3C. This antibody was re-designated MAb 458-1 and used in subsequent assays as the optimum MAb that selectively binds the C-terminal of G17. These criteria and similar assays were also applied in the selection of the MAbs raised against terminal epitopes of the gastrin hormone forms, G17, G34, G17-Gly and G34-Gly exemplified below.

The use of displacement peptides of appropriate amino acid sequence, so as to displace bound hormone, can be incorporated into assays for other gastrin hormone peptide forms to allow the amounts of both free and total (bound+free) hormone in a sample to be determined. The use of displacement peptides can also be applied to assays for total amounts of any peptide hormone for which the amino acid sequence of the region by which the peptide is bound, is available.

### EXAMPLE 3

### Isolation and Characterization of a Monoclonal Antibody to the N-terminal of Human G34

Hybridomas producing MAb to the amino terminal end of G34 were produced as described in Example 1 for the production of MAb against the C-terminal end of G17 and G34, except for the composition of the peptides used to immunize the spleen cell donor mice against the N-terminal end epitope of G34 and to select for Mab specific for the N-terminal end epitope of G34. To induce antibody response against N-terminal end epitope of G34, the peptide pELGPQGRPPPPC (SEQ ID NO: 12) was conjugated to DT to form an immunogen. This peptide was similarly linked to BSA to form the target antigen for use in the ELISA to identify Mabs against the N-terminal end epitope of G34. This fusion was designated number F401.

F401 yielded MAb 401-2. The specificity for G34 was proven by inhibition ELISA, wherein it was shown that only G34 peptide inhibited binding of the MAb 401-2 to the peptide immunomimic of the N-terminal end of G34 (SEQ ID NO: 12) as shown in Table 2.

TABLE 2
Specificity of anti-G34 MAb for gastrin isoforms¹

| | Inhibitor Concentration (nmol/ml) giving 50% inhibition³ | | | | | |
|---|---|---|---|---|---|---|
| | | | | | CCK (26-33) | |
| MAb | hG17 | eG17² | hG17-Gly | hG34³ | unsulfated | GnRH |
| 401-2 | NI | NI | NI | 0.7 | NI | NI |

¹MAb inhibition ELISA with target antigen of hG34.
²eG17 = equine G17; sequence as for human, except for Lys 7 (for Glu) and Ala 10 (for Glu).
³NI = No Inhibition.
4. Inhibitor concentration range tested was 0.01 through 100 pM.

Other forms of gastrin, including G17, G17-Gly and equine G17, as well as CCK 8 (unsulfated) and the negative control GnRH, failed to inhibit the binding of the 401-2 Mabs (as shown in Table 2).

### EXAMPLE 4

### Isolation and Characterization of Monoclonal Antibodies Against the N-terminal of G17

Hybridomas producing MAb to the amino terminal end of G17 were produced as described in Example 1 for the production of MAb against the C-terminal end of G17 and G34, except for the composition of the peptides used to immunize the spleen cell donor mice against the N-terminal end epitope of G17 and to select for Mab specific for the N-terminal end epitope of G17. To induce antibody response against N-terminal end epitope of G17, the peptide pEGPWLERPPPPC (SEQ ID NO: 5) was conjugated to DT to form an immunogen. This peptide was similarly linked to BSA to form the target antigen for use in the ELISA to identify Mabs against the N-terminal end epitope of G17. In addition, the peptide pEGPWLEEEEAAPPC (SEQ ID NO: 16) was linked to BSA to create an ELISA target antigen for the N-terminal end epitope of G17. This fusion was designated number F400. F400 yielded four MAb against the N-terminal end epitope of G17. These were designated MAb numbers 400-1 through -4.

The Mabs were produced as ascites fluid in mice by standard techniques. The ascites fluids for each of the F400 MAbs were mixed in equal volumes to form a pool of said antibodies, for use in testing. The anti-G17 MAb titer of the pool was determined by ELISA, and is shown in Table 3.

The affinity of each of the four F400 MAbs was measured by Scatchard Analysis of inhibition radioimmunoassay, wherein the binding of each MAb to radioiodinated G17 was inhibited by increasing concentrations of unlabelled G17, by standard radioimmunoassay techniques known to those skilled in the art. The affinities (Ka) of each of the MAb 400-1 through -4 are shown in Table 4. The specificity for the N-terminal end epitope of G17 was proven by inhibition ELISA, wherein it was shown that only G17, G17-Gly and equine G17 peptides inhibited binding of the MAb 400-1 through -4 to the peptide immunomimic of the N-terminal end of G17 (SEQ ID NO: 11); whereas, G34, as well as CCK 8 (unsulfated) and the negative control GnRH, failed to inhibit the binding of the 400-1 through -4 Mabs (as shown in Table 5).

Characterization of the Anti-G17 MAb

TABLE 3
Titer¹ of Anti-G17 MAb Pool, lot 012502

| MAb | ELISA titer | Specificity |
|---|---|---|
| 400-1 + 2 + 3 + 4 | 374,767 | hG17 N-terminal |

¹Established by solid phase ELISA against hG17(1-9)-“Ala”-BSA target Ag

TABLE 4
Affinity of anti-G17 MAb, # 400-1, -2, -3 and -4¹

| MAb | Ka (L/mol)² | ABC (pmol/ml)² |
|---|---|---|
| 400-1 | 1.648 × 10⁸ | 19,745 |
| 400-2 | ¹1.146 × 10¹⁰ | 8,579 |
| 400-3 | 2.820 × 10⁷ | 8,841 |
| 400-4 | 1.925 × 10⁹ | 33,650 |

¹Established using different lots of ascites fluid than those used to prepare the pool, lot # 012502. (Titers therefore likely different.)
²RIA (Scatchard analysis) with ¹²⁵I-hG17; inhibition with hG17

TABLE 5
Specificity of anti-G17 MAb for gastrin isoforms¹

Inhibitor Concentration (nmol/ml)
giving 50% inhibition⁴

| | Ab | | | | | CCK | |
|---|---|---|---|---|---|---|---|
| | Sub- | | | hG17- | | (26-33) | |
| MAb | class | hG17 | eG17² | Gly | hG34³ | unsulfated | GnRH |
| 400-1 | IgG2a | 2.03 | 1.65 | 1.79 | NI | NI | NI |
| 400-2 | IgG1 | 0.085 | 0.086 | 0.077 | NI | NI | NI |
| 400-3 | IgG1 | 1.08 | 0.12 | 1.39 | NI | NI | NI |
| 400-4 | IgG1 | 0.62 | 1.69 | 0.699 | NI | NI | NI |

¹MAb inhibition ELISA with target antigen of hG17(1-9)-“Ala”-BSA.
²eG17 = equine G17; sequence as for human, except for Lys 7 (for Glu) and Ala 10 (for Glu).
³NI = No Inhibition.
⁴Inhibitor concentration range tested was 0.01 through 100 pM.

### EXAMPLE 5

### Isolation and Characterization of Monoclonal Antibodies Against the C-terminal of Glycine-Extended G17/G34

Hybridomas producing MAb to the carboxy terminal end epitope of G17-Gly were produced as described in Example 1 for the production of MAb against the C-terminal end of G17 and G34, except for the composition of the peptides used to immunize the spleen cell donor mice against the carboxy terminal end epitope of G17-Gly and to select for Mab specific for the carboxy terminal end epitope of G17-Gly. To induce the antibody response against the carboxy terminal end epitope of G17-Gly, the peptide CPPPPSSYGWMDFG (SEQ ID NO: 14) was conjugated to DT to form an immunogen.

The peptide CGGSKKEGPWLEEEEEAYGWMDFG (SEQ ID NO: 15) was linked to BSA to form the target antigen for use in the ELISA to identify Mabs against the carboxy terminal end epitope of G17-Gly. To select for MAb that bound to G17-Gly but not to G17 or G34, the additional selective step of demonstrating MAb inhibition with G17-Gly (SEQ ID NO: 2) but with no inhibition by G17 (SEQ ID NO: 1) was employed in this fusion. This fusion was designated number F445.

F445 yielded two MAbs specific for glycine extended G17. These were designated MAb numbers 445-1 and 445-2. Creating these MAbs was especially difficult, and required that approximately 14 fusions be performed before we were successful. Normally, a single fusion is sufficient to obtain MAb to a peptide hormone, such as the other gastrin hormones described herein.

The specificity for G17-Gly was proven by inhibition ELISA, wherein it was shown that only G17-Gly peptide (SEQ ID NO: 2) and the immunogen peptide CPPPPSSYGWMDFG (SEQ ID NO: 14) inhibited binding of the MAb 445-1 and 445-2 to the G17-Gly C-terminal epitope target peptide (SEQ ID NO: 14) BSA conjugate; whereas, other forms of gastrin, including G17, G34 and equine G17, as well as CCK 8 (unsulfated) and the negative control GnRH, failed to inhibit the binding of the 445-1 and 445-2 Mabs (as shown in Table 6).

Anti-G17-Gly (C terminus) Monoclonal Antibodies (445-1,2)

Characterization of the Anti-G17-Gly MAbs

TABLE 6
Specificity of anti-G17-Gly MAbs for gastrin isoforms¹

| | | Inhibitor Concentration (nmol/ml) giving 50% inhibition³ | | | | | |
|---|---|---|---|---|---|---|---|
| | Ab | | hG17 | | | CCK | |
| | Sub- | hG17- | (12-17)- | | | (26-33) | |
| MAb | class | Gly | Gly18 | hG17² | hG34 | unsulfated | GnRH |
| 445-1 | not | 0.7 | 4 | NI | NI | NI | NI |
| | tested | | | | | | |
| 445-2 | not | 5 | 13 | NI | NI | NI | NI |
| | tested | | | | | | |

¹MAb inhibition ELISA with target antigen of hG34(16-34)-Gly 35-BSA (“Gly 16”-BSA).
²NI = No Inhibition.
³Inhibitor concentration range tested was 0.01 through 100 pM.

### EXAMPLE 6

### Isolation and Characterization of Monoclonal Antibodies Against the C-terminal of G34

Human G34 and G17 have identical C-terminal epitopes; the MAb produced in Fusion number F458, described in Example 1, yielded MAb that bind to both G34 and to G17 C-terminal end epitopes. The MAbs produced in this fusion are designated 458-1 through -5.

The specificity of MAb 458-1 through 5 for the C-terminal end epitope shared by G17 and G34 was proven by inhibition ELISA, wherein it was shown that only G17 peptide (SEQ ID NO: 1), G34 peptide (SEQ ID NO: 3) and CCK8 peptide (SEQ ID NO: 13) (which also expresses the C-terminal epitope) inhibited binding of the MAb 458-1 through 5 to the G17/34 C-terminal epitope target peptide (SEQ ID NO: 11) BSA conjugate; whereas, other forms of gastrin, including G17-Gly, G17(1-9) N terminus and the negative control GnRH, failed to inhibit the binding of the 458-1 through 5 Mabs (as shown in Table 7).

TABLE 7
Characterization of the Anti-G17, G34 (CCK 8) MAbs

| | | % Inhibition at 10 nmol/ml (250 pmoles/well)³ | | | | | |
|---|---|---|---|---|---|---|---|
| | | | | CCK | | | |
| | Ab | | | (26-33) | hG17- | | |
| MAb | Subclass | hG17 | hG34 | unsulfated | Gly | hG17(9) | GnRH |
| 458-1 | IgG1 | 94.3 | 93.5 | 93.8 | 2.1 | NI | 1.1 |
| 458-2 | Not tested | 86.0 | 84.5 | 84.8 | NI | NI | NI |
| 458-3 | not tested | 92.4 | 90.7 | 84.8 | NI | 1.2 | 1.1 |
| 458-4 | IgG1 | 88.4 | 86.7 | 83.4 | NI | NI | NI |
| 458-5 | not tested | 91.9 | 91.5 | 92.0 | NI | NI | NI |

¹MAb inhibition ELISA with target antigen of hG34(16-34)-Gly 35-BSA (“Gly 16”-BSA).
²NI = No Inhibition.
³Inhibitor concentration range tested was 0.000001 through 10 pM.

### EXAMPLE 7

### Demonstration of Anti-tumor Cell Efficacy of F400 Mabs In Vitro Against Pancreatic, Gastric and Colon Cancer Cells

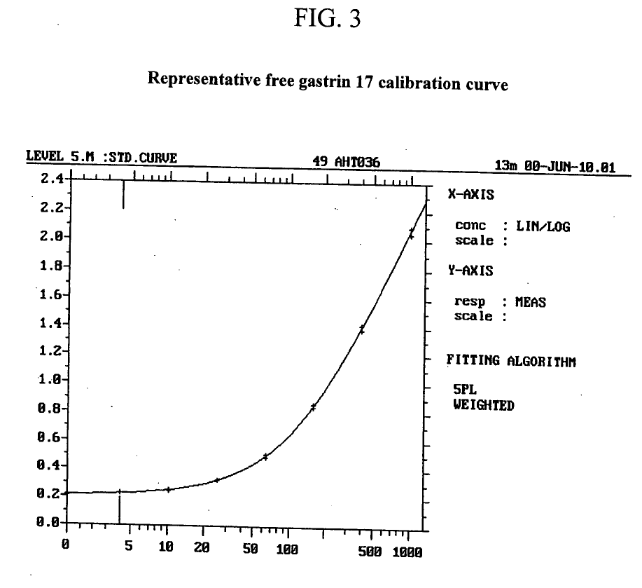

The pool of MAb to the N terminus of G17, shown in FIG. 3 of Example 4, were tested for their capacity to inhibit the growth of tumor cell lines obtained from human pancreatic, gastric and colon cancers. Two cell lines from each organ source were tested in these in vitro studies. Each of the six individual tumor cell lines tested were known to produce their own G17 hormone, potentially resulting in an autocrine effect which might be abrogated by neutralizing MAb to G17.

To prepare the F400 MAb mix for in vitro testing against cells, the antibodies were affinity purified by chromatography against peptide expressing the N-terminal epitope of G17 (SEQ ID NO: 12) linked to Sepharose (Sulfo-Link, Pierce) by methods supplied with the Sulfo-Link kit. The MAbs were dialyzed against PBS and their concentration determined by A280 measurements.

The cells were cultured under standard conditions (37 deg. C., 5% CO₂, humidified incubator). The culture media consisted of complete RPMI 1640 culture media (Gibco) containing 10% (v/v) heat inactivated fetal bovine serum (FBS, Sigma).

To harvest cells for experiments, the cells in semi-confluent monolayers were harvested with 0.025% ethylenediaminetetraacetic acid (EDTA, Sigma). The cells were washed in media and resuspended in media at a concentration of 1×10⁵ viable cells/mL and plated into 96 well culture plates at 0.1 mL/well. After overnight incubation, the medium was aspirated out and replaced with fresh culture media containing 500 μg/mL of either the mixture of F400 MAbs or with normal mouse immunoglobulin (NMIg). The cells were then incubated for a further 48 hours, following which the cell proliferation was assessed by the tetrazolium-based MTT assay commonly used to assess cell growth in in vitro cultures of mammalian cells. The absorbance of each well obtained from the MTT assay were averaged for each test group (n=5). The percent to which the F400 MAbs inhibited cell growth relative to growth in the presence of NMIg was then calculated.

The results of these tests are given in Table 8, which shows that the anti-G17 MAb mix inhibited the growth of each tumor cell line tested. Inhibition ranged from 19.5% for a pancreatic tumor cell line to 52.0% against a gastric cell line.

Thus, it was shown that the MAb of this invention had anti-growth therapeutic activity in vitro against tumors from three common malignancies of the gastrointestinal tract.

TABLE 8
Basal Growth Inhibition of Six Human Tumor Cell Lines by
anti-G17 MAb Mixture of 400-1, -2, -3, -4.

| Cancer | Pancreatic | | Gastric | | Colon | |
|---|---|---|---|---|---|---|
| Type | Cancer | | Cancer | | Cancer | |
| Cell Line | BxPC3 | PAN-1 | MGLVA1 | ST16 | C170HM2 | HCT116 |
| % | 19.5 | 22.0 | 40.0 | 52.0 | 50.0 | 41.0 |
| Inhibition | | | | | | |
| of Cell | | | | | | |
| Growth | | | | | | |
| by F400 | | | | | | |
| MAb Mix | | | | | | |

### EXAMPLE 8

### Demonstration of Anti-tumor Efficacy of F400 Mabs In Vivo Against Gastric Cancer Cells

The pool of MAb to the N terminus of G17, containing an equal volume mixture of ascites fluid containing each of MAbs 400-1 through -4, were tested for their capacity to inhibit the growth of a tumor cell line obtained from a human gastric cancer, MGLVA1. MGLVA1 cells are known to produce their own G17 hormone, potentially resulting in an autocrine effect which might be abrogated by neutralizing MAb to G17.

To prepare the F400 MAb ascites fluid mix for in vivo testing, the ascites fluid was depleted of complement by heating the ascites fluid at 56° C. for 30 minutes. A negative control ascites fluid, purchased from Sigma, was similarly treated.

MGLVA1 gastric cancer cells were grown as subcutaneous tumors in female nude mice. To implant tumors in the test mice, tumors were surgically removed from tumor-bearing mice and cut into pieces about 1 mm³. These fragments were then implanted subcutaneously into the flanks of nude mice to be used in the study, and the tumors were allowed to take. The tumor sites were observed and tumor growth measured with calipers. When the tumors were observed to take, the mice were randomized into groups to be treated with the test MAb (F400 mix) or with the negative control ascites. There were 12 mice/group in the study.

In the first week, the mice were injected with 0.2 mL of ascites fluid (either F400 mix or the negative control), intraperitoneally, twice weekly. After the first week, the injection volume was reduced to 0.1 mL, twice weekly. Tumors were measured 3 times per week. The study ran for 27 days. At the end of the study, the mice were sacrificed and the tumors were excised and weighed.

The mean weight of the MGLVA1 gastric cancer tumors in mice treated with the F400 test MAb mix was 0.75 g whereas the mean weight of tumors from mice bearing MGLVA1 gastric cancer tumors and treated with the ascites fluid as a negative control was 1.5 g. Thus, the anti-G17 MAbs from the F400 test mix exerted a strong growth inhibitory effect on the gastric cancer cells, reducing the tumor weights by 50%.

### EXAMPLE 9

### ELISA for the Determination of the Titer of Antibodies to the C-terminus of G17 and G34

The purpose of this analytical method is to determine the titer of anti-hG17 antibodies in test serum by ELISA. Briefly, the anti-hG17 antibody ELISA of the invention is based upon the specific binding of antibodies (Ab, either polyclonal or monoclonal) to hG17 epitopes expressed by the hG17₍₁₋₉₎-AAPPC-BSA conjugate (amino acids 1-9 of human gastrin hormone peptide coupled to BSA through the linker of SEQ ID NO: 16).

In the first step, conjugate was bound to the wells of a 96 well ELISA plate. Free conjugate was removed by a wash step using a 96 well plate washer. The test (or control) antiserum was then added. Anti-hG17 Ab present in the test serum bound to the conjugate by virtue of hG17 peptide epitopes present on the antigen. The antibodies were then detected by the addition of an anti-IgG-Alkaline Phosphatase reagent, which is species specific for the anti-hG17 antibodies being detected. For example, rabbit anti-hG17 antibodies are detected using Goat anti-Rabbit IgG-Alkaline Phosphatase conjugate (“GAR-AP”), which binds to the rabbit anti-hG17 Ab, as the Ab detection reagent. The AP moiety of anti-Ig-AP conjugate subsequently catalyses conversion of substrate to a colored product (p-nitrophenol). Color development was measured as absorbance at 405 nm in an ELISA plate reader.

A standard serum containing pooled anti-hG17 serum or ascites fluid containing anti-hG17 MAb with an assigned reference titer, from the same animal species as test samples was used as positive control. Serum from the same animal species as the test sample e.g., normal sera, pre-immune sera, etc. was used as negative control.

The magnitude of color development in the linear range was directly proportional to the quantity of anti-hG17 Ab bound to the target antigen. A plot of the dilution series of the positive standard (anti-hG17) serum versus absorbance values was used to generate a standard curve. The anti-hG17 Ab titers of the test samples were then determined from the dilution that produces the same absorbance as the reference titer of the positive standard (e.g., 1:200,000 dilution of rabbit anti-hG17 positive standard).
REAGENT SOLUTIONS: The quantities of reagents and solutions specified for preparation in this analytical method are only for convenience. The actual quantities can be scaled according to requirements.
1. Carbonate buffer with 0.02% NaN₃ (“Carbonate buffer”): Made by dissolving 1.59 g Na₂CO₃ and 2.93 g NaHCO₃ in approximately 750 ml of distilled water with a magnetic stirrer. Add 4 ml of 5% NaN₃ solution and stir. Adjust to 1.0 liter with water. Measure the pH, which should be 9.6±0.2 (if necessary, adjust the pH with 1.0 M NaOH or 1.0 M HCl). Store in the refrigerator until needed.
2. FTA (PBS) with 0.05% Tween-20 and 0.02% NaN₃ (“FTA/Tween”): Dissolve 9.23 g FTA in approximately 750 ml of purified water. Add 0.5 ml Tween-20 and 4 ml 5% NaN₃. Adjust to 1.000 liter with water.
3. 1% BSA in FTA/Tween (“BSA/FTA/Tween”): Dissolve 10 g BSA in 1000 ml FTA/Tween.
4. Substrate buffer: Dissolve 50 mg MgCl₂.6H2O in 448 ml of purified water. Add 50 ml of DEA and 2 ml 5% NaN₃. Adjust the pH to 9.8 with concentrated HCl. Store protected from light at room temperature.
5. PBS, pH 7.2: Can be prepared from solid FTA (FTA Hemagglutination Buffer (“FTA”) (Becton Dickenson Microbiology Systems, Cockeysville, Md.)).
ELISA PROCEDURE: Coating with Antigen: A solution of 1 μg/ml hG17₍₁₋₉₎-AAPPC-BSA conjugate (amino acids 1-9 of human gastrin hormone peptide coupled to BSA through the linker of SEQ ID NO: 16). in Carbonate buffer is prepared. A minimum of 5.2 ml of antigen solution is needed for each plate to be coated. Antigen solution is prepared by making a 1:1000 dilution of the 1 mg/ml conjugate stock solution with Carbonate buffer. Plates may be any plate suitable for ELISA assays, such as for instance, Microtiter® Immunoassay Plates, rigid styrene (e.g., Immulon® 2 “U” bottom 96 well plates, Dynatech Laboratories, Inc., VA; or Flat-bottom 96 well plates, polystyrene: e.g., Microwell Plates, NUNC, vendor VWR). Immulone 2 “U” bottom plates are coated with antigen by adding 50 μl/well of the antigen solution. Plates are stored in a moist chamber (e.g., a closed container with a moist paper towel) to prevent moisture loss and incubated overnight in the refrigerator (at 2°-8° C.).

PREPARATION OF SERUM DILUTIONS: 1/10^{0.5} serial dilution series of the positive standard and negative control and test sera were prepared as shown in Table 9. Sera were diluted in BSA/FTA/Tween solution in flat bottom 96 well plates (12-channel multipipettors enable simultaneous dilution of up to 12 sera).

TABLE 9
Serial dilutions starting at 1:1000 were prepared as shown

| 96 well plate | Serum | Titer¹ |
|---|---|---|
| Row # | Dilution | (= 1/Dilution) |
| A | 1:1,000 = 10⁻³ | 10³ |
| B | 1:3,162 = 3.16 × 10⁻⁴ = 10^{-3.5} | 3.16 × 10³ |
| C | 1:10,000 = 10⁻⁴ | 10⁴ |
| D | 1:31,623 = 3.16 × 10⁻⁵ = 10^{-4.5} | 3.16 × 10⁴ |
| E | 1:100,000 = 10⁻⁵ | 10⁵ |
| F | 1:316,230 = 3.16 × 10⁻⁶ = 10^{-5.5} | 3.16 × 10⁵ |
| G | 1:1,000,000 = 10⁻⁶ | 10⁶ |
| H | 1:3,163,300 = 3.16 × 10⁻⁷ = 10^{-6.5} | 3.16 × 10⁶ |

¹The titer of each dilution is calculated as the reciprocal of the dilution.

A sufficient volume of a dilution of each serum was prepared to provide a minimum working volume of 200 μl. Depending on the serum titer, dilutions begining with a 1/100 (for low titer serum) or 1/1000 (for high titer serum) dilution of each serum in row A were made, then proceeding with serial dilutions down each column to row H (See Table 9), yielding a total of eight dilutions of each sample. The dilution series of the negative control was prepared beginning at 1/100. Samples of the dilution series of the positive standard serum and the prebleed/negative control serum were run in duplicate on each plate.
PLATE WASHING: Using the plate washer, (e.g., Ultrawash Plus; or, DynaWasher II (Dynatech Laboratories, Inc., VA) or equivalent) the coated plates were washed four times each with FTA/Tween and then “slapped” the plates on paper towels to remove residual solution.

ANTIBODY BINDING: Following the sample plate dilution series as shown in Table 10 below, 50 μl/well of the diluted serum was transferred to the corresponding wells of the antigen coated “U” plates. The plates were incubated in a moist chamber for 1 hour at room temperature.

TABLE 10
EXAMPLE OF A 96 WELL PLATE ELISA SETUP

| | | | | | | | | | | | | | Sample |
|---|---|---|---|---|---|---|---|---|---|---|---|---|---|
| | 1 | 2 | 3 | 4 | 5 | 6 | 7 | 8 | 9 | 10 | 11 | 12 | Dilution |
| A | Neg. | Neg. | Pos. | Pos. | TS1 | TS2 | TS3 | TS4 | TS5 | TS6 | TS7 | TS8 | 10⁻³ |
| B | Neg. | Neg. | Pos. | Pos. | TS1 | TS2 | TS3 | TS4 | TS5 | TS6 | TS7 | TS8 | 10^{-3.5} |
| C | Neg. | Neg. | Pos. | Pos. | TS1 | TS2 | TS3 | TS4 | TS5 | TS6 | TS7 | TS8 | 10⁻⁴ |
| D | Neg. | Neg. | Pos. | Pos. | TS1 | TS2 | TS3 | TS4 | TS5 | TS6 | TS7 | TS8 | 10^{-4.5} |
| E | Neg. | Neg. | Pos. | Pos. | TS1 | TS2 | TS3 | TS4 | TS5 | TS6 | TS7 | TS8 | 10⁻⁵ |
| F | Neg. | Neg. | Pos. | Pos. | TS1 | TS2 | TS3 | TS4 | TS5 | TS6 | TS7 | TS8 | 10^{-5.5} |
| G | Neg. | Neg. | Pos. | Pos. | TS1 | TS2 | TS3 | TS4 | TS5 | TS6 | TS7 | TS8 | 10⁻⁶ |
| H | Neg. | Neg. | Pos. | Pos. | TS1 | TS2 | TS3 | TS4 | TS5 | TS6 | TS7 | TS8 | 10^{-6.5} |

Abbreviations:
Pos. = Positive standard serum;
Neg. = Prebleed/negative control serum;
TS1-TS8 = Test Sera

ANTIBODY DETECTION REAGENT: An appropriate dilution of Anti-Ig-Alkaline Phosphatase Conjugate was prepared in FTA/Tween. A minimum of 5.2 ml per plate in the assay was required. Plates were washed as described above. 50 μl/well of the GAR-AP solution (Anti-Ig-Alkaline Phosphatase Conjugate e.g., for testing rabbit anti-hG17 antibodies, Goat anti-Rabbit IgG (H+L)-Alkaline Phosphatase (Antibodies Inc., Davis, Calif.)) was added to every well in the “U” plate and incubated at room temperature in the moist chamber for 1 hour.

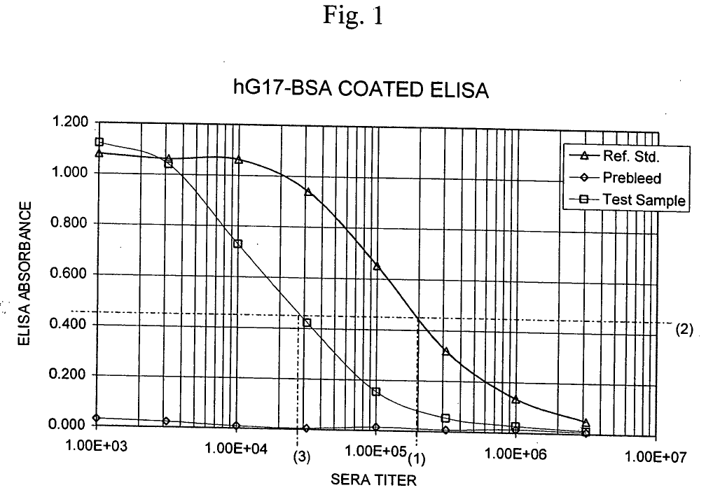

To detect anti-hG17 antibodies in serum obtained from species other than rabbit, an anti-Ig-AP conjugate must be used that is specific for the species that produced the test serum (e.g., human anti-hG17 antibodies would be detected with an anti-human IgG-AP reagent, used at the dilution established for each lot of reagent). The positive standard and negative control serum should be obtained from the same species as the test serum.
SUBSTRATE SOLUTION: p-NPP tablets (p-nitrophenylphosphate, supplied as Phosphatase Substrate Tablets, Sigma 104 (“p-NPP”) (Sigma Chemical Co., St. Louis, Mo.)) were removed from the freezer and allowed to warm to room temperature. Immediately before use, a 1 mg/ml solution of p-NPP was prepared by adding 1 tablet of p-NPP to 5 ml of DEA substrate buffer (at room temperature). Each 5-ml aliquot of substrate solution was sufficient for 1 assay plates. Substrate solution was stored in the dark until used.
SUBSTRATE ADDITION: Plates were washed as described above. To all wells, beginning with column 1, 50 μl/well of p-NPP solution was simultaneously added with an 8 (or 12) channel multipipettor beginning with row A.
MONITORING REACTION: The development of the substrate solution was stopped when the absorbance of the dilution of the positive standard nearest the reference titer reached 10-30% of the ELISA plate reader's maximum linear reading range. The ELISA plate reader such as an MRX Automated Plate Reader; or, MR 580 MicroELISA Auto Reader (Dynatech Laboratories, Inc., VA); or equivalent was used to monitor the absorbance of the positive standard to determine when the dilution corresponding to the reference titer reaches 10-30% of the reader's range (usually after 10-30 minutes of development time). The ELISA reader was set to measure at A₄₀₅ nm for p-nitrophenol.
STOP REACTION: When the selected dilution of the positive standard above gave an absorbance value of 10-30% of the reader's linearity range, the reaction was stopped by adding 50 μl of 1.0 M NaOH to each well with the 8 (or 12) channel pipettor. The NaOH solution was added to the wells in the same order, and with the same timing, as the substrate solution was added. The reagents were gently mixed by carefully shaking the plate on the counter top.
MEASURE ABSORBANCE: The entire plate was read with an ELISA reader.
DATA ANALYSIS: The titer of each serum was determined as follows: The absorbance obtained for the negative control serum was subtracted from the absorbance of each corresponding dilution of positive standard and test serum. (The mean values for each dilution for the positive standard and the negative control were used.) The absorbance was plotted on the ordinate (linear scale) against (1/dilution) on the abscissa (log scale) for each serum, including the positive standard, on a semi-log graph scale. By plotting the inverse of the dilution, the titer could be read directly on the X-axis. Occasionally, an absorbance value was clearly off the binding curve for a particular serum (outlier points); such values were excluded from the curve. The titer of each serum is determined as the reciprocal of the dilution that yields the same absorbance as that produced by the reference titer of the positive standard (e.g., 1:200,000 dilution of rabbit anti-hG17 positive standard). An example of the data analysis is provided FIG. 1.

### EXAMPLE 10

### Determination of Antibody Specificity by Inhibition ELISA

The same method as in the Example above is followed for the peptide inhibition ELISA with the exceptions described below.
PREPARATION OF INHIBITOR: The appropriate target hormone peptide, in this case hG17, is prepared to a working stock of 1 μmol/ml (1000 μM). The inhibition dilution series was prepared from the working stock solution, at dilution ratios from 1:2 to 1:10, yielding a total of 8 dilutions or 12 depending on the layout on the plate.

Preparation of Sample Dilution: Titration series of the samples are done prior to the inhibition assay to establish the dilution of the antibody sample at 50% maximal binding. The sample was then prepared to 2× the 50% binding concentration, for mixing with equal volumes of peptide inhibitor and with buffer as a control in the inhibition assay. The sample mixture was incubated in a moist chamber for approximately 30 minutes and then added to the washed coated ELISA plate and incubated for approximately 1 hour in a humidity chamber.

The percent binding was determined from the absorbance readings (subtracted from the background) by dividing the absorbance obtained from the sample with inhibitor by the absorbance obtained from the sample control without inhibitor, and multiplying this value by 100. Finally, the percent inhibition was determined by subtracting the percent binding from 100%.

The test samples can be serum, MAb in cell culture supernatant, ascites fluid, or affinity-purified antibody (Ab). For Abs against target antigens other than the amino terminus of G17, the appropriate target hormone antigen and inhibitor are used. An unrelated peptide should be included as a negative control.

ELISA: Data Analysis

FIG. 1 shows an example of the data obtained with the ELISA described above. The mean negative control serum absorbance values were subtracted from the mean positive standard and test serum values to obtain the net absorbance values at each dilution. The net absorbance values were plotted against the titer. (In the example, the negative control is also plotted to demonstrate typical values.)

Gastrin-17 Stability

The stability of Gastrin at room temperature (about 22° C.) was assessed by the total gastrin assay as described above by measuring total G17 immediately after sample preparation to achieve known G17 concentrations of 15, 100 and 600 pM, and after 2 hours at room temperature on the bench. The results, demonstrating a substantial decrease in G17 concentration in each of the samples, are shown in Table 11, below.

TABLE 11
Total Gastrin 17 assay
Stability of gastrin 17 in human plasma at room temperature
(ca 22° C.)

| | | Measured gastrin 17 | | |
|---|---|---|---|---|
| | | concentration (pM) | | |
| | | 15 | 100 | 600 |
| 0^{a} hours | mean | 11.6 | 89.4 | 605.5 |
| | sd | 2.8 | 4.3 | 25.0 |
| | CV(%) | 23.8 | 4.8 | 4.1 |
| | RE(%) | −22.7 | −10.6 | 0.9 |
| 2 hours | mean | 5.5 | 59.1 | 400.5 |
| | sd | 3.1 | 2.0 | 19.7 |
| | CV(%) | 55.2 | 3.5 | 4.9 |
| | RE(%) | −63.3 | −40.9 | −33.3 |

^{a}Mean result used as baseline
sd Standard deviation
CV Coefficient of variation (calculated before rounding)
RE Relative error (calculated after rounding)

### EXAMPLE 11

### Inhibition Radioimmunoassay (RIA) of Antiserum to HG17—Serum Titration and Antigen Inhibition RIA for the Determination of the Antigen Binding Capacity (ABC) of Anti-human Gastrin 17 (hG17) Antisera

Dilution Buffers:

1. Phosphate buffered saline, pH 7.2 (PBS)+0.02% sodium azide (NaN₃). The commercial preparation of soluble solids, “FTA Hemagglutination Buffer” can be dissolved in distilled water to produce PBS (9.23 g/l gives a solution of pH 7.2±0.1).
2. FTA with 1% bovine serum albumin (BSA) and 0.02% NaN₃.
3. Supplemented calf serum (SCS; GIBCO), stored frozen in aliquots, 50 ml or smaller.
4. PEG, MW 8000, made up as a 25% solution, (250 g per liter; dissolves slowly). Store at 4° C.
5. Human Gastrin 17 (15-Leu) (Research Plus, # 07-027-002); in single use aliquots at 5-10 μg/ml in FTA/1% BSA/azide. Stored at −70° C.
6. Human gastrin 17-¹²⁵I (NEN).
   Methods

The test sera were first titrated against a set of quantity of hG17-¹²⁵I to establish the volume of each antiserum to be tested in the inhibition RIA. The sera were then tested by inhibition RIA, and the antigen binding capacity (ABC) calculated by Scatchard Analysis.

Titration RIA Protocol

1. For the positive control antiserum and all test antisera, duplicate tubes were set up for each antiserum dilution to be assayed; preferably five 10-fold dilutions of serum were made, so that the final dilutions in the assay tubes were 1:40 to 1:400,000. This is equivalent to a range of 10 μl to 0.001 μl of antiserum added per tube.
2. 300 μl of dilution buffer was dispensed into two tubes that served as reagent blanks; 200 μl dilution buffer was added to all remaining tubes.
3. 100 μl of diluted antiserum was transferred into each tube of serum duplicates (2×100 μl of each dilution is needed). Starting with 30 μl antiserum for the dilution series (yielding 300 μl of a 1:10 dilution) was sufficient to allow for transfer losses.
4. At least one dilution (1:40, the lowest dilution of test sera) of a negative sera was included as nonspecific binding control.
5. ¹²⁵I-labeled antigen (Ag) that was diluted in RIA buffer to about 10,000 cpm/0.1 ml. See dilution procedure below.
6. 100 μl of labeled gastrin was added to all tubes. 100 μl of labeled hG17 was added to ten scintillation vials or gamma-counting tubes to establish total counts added.
7. Tube contents were mixed by shaking or vortexing and covered with parafilm.
8. Tubes were incubated at 4° C., overnight, ˜18 hours. This is the minimum incubation period: longer incubations can be used in the Titration RMA but this is not usually necessary.
9. 100 μl SCS was added to all tubes and the tubes were shaken.
10. 500 μl of 25% PEG (4° C. or RT) was added to all tubes and vortexed to mix.
11. Tubes were centrifuged at 2000×g for 30 minutes, at 4-12° C.
12. Supernatants were aspirated and discarded from all tubes.
13. Precipitates were counted in assay tubes in a gamma counter or prepared for scintillation counting, as described below.
   Calculations

Duplicate sample counts per minute (cpm) were averaged. The nonspecific background binding was not subtracted. The data was plotted: % hG17-¹²⁵I bound vs. volume of serum added. The amount of each serum that binds 35% of the total cpm added per sample was chosen for the inhibition RIA.

Inhibition RIA

1. One dilution of antiserum was used for each inhibition series, as determined in the titration RIA. The number of duplicate tubes to set up was established by the number of dilutions of inhibitor to be tested, including uninhibited controls. Typically, 8 dilutions of inhibitor (16 tubes) plus 2 uninhibited tubes were run per antiserum.
2. 300 μl dilution buffer was dispensed into two tubes as zero-count blanks (to establish the natural background counts).
3. 200 μl buffer was dispensed to six tubes to receive negative control serum (for background nonspecific binding); two of these were run at the end of the assay. 200 μl buffer was dispensed to two tubes for each antiserum (for total counts bound). These tubes did not receive any hG17 inhibitor.
4. 100 μl dilution buffer was added to all remaining tubes.
5. 100 μl of unlabeled hG17 (inhibitor), diluted to give the proper final concentrations (see below), was dispensed into duplicate tubes for each test and control antiserum. These series established the hG17 inhibition curves for each antiserum. The hG17 inhibitor was prepared by a 1:1 dilution series starting with 5120 pg/0.1 ml in FTA/azide.
8. The tubes were mixed.
9. The ¹²⁵I-labeled antigen in RIA buffer was diluted to approximately 10,000 cpm/0.1 ml.
10. 100 μl of hG17-¹²⁵I was added to all tubes, including twelve or more tubes spaced throughout the assay, to establish the total counts added.
11. Lastly, 100 μl of appropriately diluted anti-hG17 control serum, negative control serum, or test serum was added to each tube of the appropriate tube sets.

12. The tubes were mixed and covered (e.g., with parafilm).

TABLE 12
Summary of Setup

| | | Volume (in μl)/Tube | | | | |
|---|---|---|---|---|---|---|
| | | | | | Test | |
| | | | | | antiserum | |
| | | | | | or positive | negative |
| | | | | hG17 | control | control |
| Series | #Tubes | Buffer | hG17-¹²⁵I | Inhibitor | serum | serum |
| A. | 2 | 300 | 100 | — | — | — |
| B. | 2n | 200 | 100 | — | 100 | — |
| C. | 6 | 200 | 100 | — | — | 100 |
| D. | 2i | 100 | 100 | 100 | 100 | — |

A. Nonspecific background control without serum.
B. Total counts bound. n = number of antisera
C. Nonspecific background with negative control serum.
D. hG17 inhibition series. i = number of inhibitor concentrations

13. Tubes were incubated at 4° C. for ˜42 hours (two days).
14. 100 μl SCS was added to all tubes and mixed.
15. 500 μl of 25% PEG was added to all tubes and mixed.
16. Tubes were centrifuged at 2,000×g for 30 minutes, at 4° C.
17. The supemate was aspirated and discarded from all tubes.
18. The precipitates were counted in the assay tubes in a gamma counter or prepared for scintillation counting. For scintillation counting 250 μl of dH₂O was added to all assay tubes; heating the water to 90-100° C. speeds pellet dissolution, which required 2-3 hours. 3 ml scintillation fluid was then added to each of the scintillation vials. All of the dissolved pellet was transferred from a single tube to a scintillation vial and placed in racks for counting.
   Calculations
1. Duplicate sample cpm were averaged and the nonspecific background binding (determined by average of the negative serum controls) was subtracted.
2. Total counts were added and baseline background controls were used to ensure that the total counts bound by uninhibited anti-HG17 antibody were in the range expected.
3. Using the total counts added and the counts bound for each quantity of inhibitor, the ABC and affinity constant of the antisera were determined by Scatchard analysis (plot of bound/free versus bound antigen). For each individual antiserum, the points that gave the best linear regression line were chosen, the rest were deleted. This was done by viewing the plot and noting the regression coefficient. Generally the lower section of the plot was not used. The ABC and affinity constant were calculated automatically by the spreadsheet set up for this purpose.
   Dilution of HG17-¹²⁵I

The source of hG17-¹²⁵I was NEN. The radiolabeled hormone (15 μCi) had a specific activity of 2200 μCi/mmole when shipped. Following the accompanying package instructions, the lyophilate was diluted to 50 μCi/ml with dH₂O, based on the number of days decay. After dissolving, 50 μl aliquots were made and stored at −70° C. in a lead container.

Dilution to 10,000 cpm

Each assay tube (0.1 ml) received approximately 10,000 cpm of labeled compound. (Normally 10,000-10,400 cpm/tube.) When determining the volume of diluted hG17-¹²⁵I needed, an extra 3-4 ml was allowed for total count determinations and losses from transfer and foaming.

Note: The test samples to be run in this assay may be serum, MAb in cell culture supernatant, ascites fluid, or affinity-purified Ab. For antibodies against target antigens other than the amino terminus of G17, the appropriate ¹²⁵I-labeled target hormone antigen and inhibitor are used. A non-related peptide should be included and tested as a negative control.

### EXAMPLE 12

### Detection of CCK 2 Receptor on Paraffin Embedded Tissue with Rabbit α-GRE 11 Antibodies

Tissue sections were deparaffmized by submersion in 3 separate xylene baths (5-6 dips, each bath) and then rehydrated by incubation in 100% industrial methylated spirits (IMS) alcohol (5-6 dips, each bath). Slides were rinsed in distilled water for 5 mins. Endogenous alkaline phosphatase activity was blocked by incubating the slides for 20 mins in 15% acetic acid. The slides were then rinsed in distilled water for 5 min. The slides were placed in a plastic slide rack two spaces apart from each other and microwaved at full power (600 W) for 10 mins in citrate buffer, pH 6 (2.1 g citric acid monohydrate, z 12.5 mL 2M NaOH per 1 L), making sure that there was sufficient buffer to cover the slides for the entire processing time. Slides were then immediately transferred to cold, running, distilled water for 3-4 min. taking care not to allow the slides to dry out.

Sections were marked using a hydrophobic pen, placed in a humidifying chamber and soaked in TRIS buffered saline (TBS), pH 7.6, for 5 min., (0.66 g TRIS-(hydroxymethyl) methylamine, 8.75 g NaCl,≈4.15 mL HCL), at room temperature (“RT”). Non-specific binding of the secondary antibody (“Ab”) was blocked by incubating the slides in 10% Normal Goat Serum in TBS for 20 min. at RT. The slides were drained and primary antibody was added to each slide, (200 μL/slide), and left for 1 hour at RT in a humidifying chamber. The slides were washed by first gently rinsing with TBS, (in a squirt bottle; taking care not to aim the stream directly at the tissue section), and then soaked in buffer for 5 min.

Alkaline phosphatase-conjugated goat anti-rabbit secondary antibody (or appropriate antibody targeted to the source of the test antibody) was added to the slides at a 1/50 dilution in TBS, 200 μL/slide. The slides were then incubated for 1 hour at RT and then washed in TBS for 5 mins.

Fast red substrate, (Vector Red, Vector Labs/Fast Red, Sigma), was prepared just prior to use and added to each section for a maximum time of 20 mins (Vector) or 30 mins (Sigma). Slides were rinsed in TBS, distilled water and then counterstained in Mayer's Haematoxylin (times vary). After staining, the slides were transferred to distilled water.

Slides were then dipped in 1% acid alcohol, (10 mL conc HCL, 700 mL IMS, 290 mL distilled water per liter), to remove excess counterstain, except if Fast Red substrate (Sigma) was used, and then transferred to distilled water. Slides were dipped in 0.5% sodium tetraborate solution (diluted in water) several times. At this point it is advisable to check one of the sections under the microscope to see if the nuclei are blue as opposed to purple. Stained slides were then transferred to distilled water, followed by IMS and finally xylene before mounting with DPX (xylene mountant).

The appropriate dilutions of test and control sera/purified antibody were established using a dilution series. GI sections are best stained with the alkaline phosphatase reagent system. Although ABC is more specific, it brings out a lot of non-specific staining due to its high sensitivity. Intestinal alkaline phosphatase can be blocked with Levamisole (Vector labs), which is added to the substrate solution when developing the sections. Vector red substrate is less problematic than the Sigma substrate product. However, it is necessary to add a drop of Levamisole (Vector Labs) to the prepared substrate solution.

The primary antibody (Ab) used in this immunohistochemical method can be serum, MAb, Ab in cell culture supernatant, ascites fluid, or affinity-purified Ab.

The skilled artisan will immediately recognize that the procedures described herein above can be applied to the isolation of optimal MAbs for immunodetection and immunoenzymometric assays of other peptides, particularly other hormone peptides, including other gastrin hormone forms. The present invention contemplates the full scope of the MAbs as taught and exemplified by the non-limiting examples described herein. All of the patents and publications cited in this specification are hereby expressly incorporated by reference in their entireties.

Deposit of Hybridoma Cell Lines

The following hybridomas that produce particular MAbs of the present invention were deposited with the American Type Culture Collection (ATCC, Manassas, Va.) on Mar. 25, 2004:
1. Hybridoma 400-1 producing MAb 400-1 was assigned accession number ______.
2. Hybridoma 400-2 producing MAb 400-2 was assigned accession number ______.
3. Hybridoma 400-3 producing MAb 400-3 was assigned accession number ______.
4. Hybridoma 400-4 producing MAb 400-4 was assigned accession number ______.
5. Hybridoma 401-2 producing MAb 401-2 was assigned accession number ______.
6. Hybridoma 445-1 producing MAb 445-1 was assigned accession number ______.
7. Hybridoma 445-2 producing MAb 445-2 was assigned accession number ______.
8. Hybridoma 458-1 producing MAb 458-1 was assigned accession number ______.

What is claimed is:

1. A monoclonal antibody that selectively binds the N-terminus of gastrin-17 (G17) at an epitope within the amino acid sequence pEGPWLE (SEQ ID NO: 5).

2. The monoclonal antibody of claim 1, wherein the antibody has an association constant for human gastrin-17 (hG17) of from about 10⁷ LM⁻¹ to about 10⁸ LM⁻¹.

3. The monoclonal antibody of claim 1, wherein the antibody has an association constant for human gastrin-17 (hG17) of from about 10⁸ LM⁻¹ to about 10⁹ LM⁻¹.

4. The monoclonal antibody of claim 1, wherein the antibody has an association constant for human gastrin-17 (hG17) of from about 10⁹ LM⁻¹ to about 10¹⁰ LM⁻¹.

5. The monoclonal antibody of claim 1, wherein the antibody has an association constant for human gastrin-17 (hG17) of from about 10¹⁰ LM⁻¹ to about 10¹¹ LM⁻¹.

6. The monoclonal antibody of claim 1, wherein the antibody has the binding characteristics of the monoclonal antibody produced by the hybridoma selected from the group consisting of 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

7. The monoclonal antibody of claim 6, wherein the monoclonal antibody is humanized.

8. The monoclonal antibody of claim 6, which is the antibody produced by the hybridoma selected from the group consisting of 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

9. A monoclonal antibody that selectively binds the C-terminus of gastrin-17 (G17) and gastrin-34 G34) at an epitope within the amino acid sequence KKEGPWLEEEEEAYGWMDF-NH₂ (SEQ ID NO: 6).

10. The monoclonal antibody of claim 9, wherein the antibody has the binding characteristics of the monoclonal antibody produced by the hybridoma 458-1 (ATCC accession number PTA-5896).

11. The monoclonal antibody of claim 10, wherein the monoclonal antibody is humanized.

12. The monoclonal antibody of claim 10, which is the antibody produced by the hybridoma 458-1 (ATCC accession number PTA-5896).

13. A monoclonal antibody that selectively binds the N-terminus of human gastrin-34 (G34) at an epitope within the amino acid sequence pELGPQG (SEQ ID NO: 7).

14. The monoclonal antibody of claim 13, wherein the antibody has the binding characteristics of the monoclonal antibody produced by the hybridoma 401-2 (ATCC accession number PTA-5893).

15. The monoclonal antibody of claim 14, wherein the monoclonal antibody is humanized.

16. The monoclonal antibody of claim 14, which is the antibody produced by the hybridoma 401-2 (ATCC accession number PTA-5893).

17. A monoclonal antibody that selectively binds the C-terminus of glycine-extended gastrin-17 (G17-Gly) and glycine-extended gastrin-34 (G34-Gly) at an epitope within the amino acid sequence YGWMDFG (SEQ ID NO: 8).

18. The monoclonal antibody of claim 17, wherein the antibody has the binding characteristics of the monoclonal antibody produced by the hybridoma 445-1 (ATCC accession number PTA-5894).

19. The monoclonal antibody of claim 18, wherein the monoclonal antibody is humanized.

20. The monoclonal antibody of claim 18, which is the antibody produced by the hybridoma 445-1 (ATCC accession number PTA-5894).

21. The monoclonal antibody of claim 17, wherein the antibody has the binding characteristics of the monoclonal antibody produced by the hybridoma 445-2 (ATCC accession number PTA-5895).

22. The monoclonal antibody of claim 21, wherein the monoclonal antibody is humanized.

23. The monoclonal antibody of claim 21, which is the antibody produced by the hybridoma 445-2 (ATCC accession number PTA-5895).

24. A panel of monoclonal antibodies, comprising an antibody that selectively binds the N-terminus of gastrin-17 (G17) at an epitope within the amino acid sequence of SEQ ID NO: 5 and an antibody that selectively binds the C-terminus of gastrin-17 (G17) or gastrin-34 (G34) at an epitope within the amino acid sequence of SEQ ID NO: 6.

25. A panel of monoclonal antibodies, comprising an antibody that selectively binds the N-terminus of gastrin-17 (G17) at an epitope within the amino acid sequence of SEQ ID NO: 5 and an antibody that selectively binds the C-terminus of glycine-extended gastrin-17 (G17-Gly) and glycine-extended gastrin-34 (G34-Gly) at an epitope within the amino acid sequence of SEQ ID NO: 8.

26. A panel of monoclonal antibodies, comprising an antibody that selectively binds the N-terminus of human gastrin-34 (G34) at an epitope within the amino acid sequence of SEQ ID NO: 7 and an antibody that selectively binds the C-terminus of gastrin-17 (G17) or gastrin-34 (G34) at an epitope within the amino acid sequence of SEQ ID NO: 6.

27. A panel of monoclonal antibodies, comprising an antibody that selectively binds the N-terminus of human gastrin-34 (G34) at an epitope within the amino acid sequence of SEQ ID NO: 7 and an antibody that selectively binds the C-terminus of glycine-extended gastrin-17 (G17-Gly) and glycine-extended gastrin-34 (G34-Gly) at an epitope within the amino acid sequence of SEQ ID NO: 8.

28. Hybridoma 400-1 (ATCC accession number PTA-5889).

29. Hybridoma 400-2 (ATCC accession number PTA-5890).

30. Hybridoma 400-3 (ATCC accession number PTA-5891).

31. Hybridoma 400-4 (ATCC accession number PTA-5892).

32. Hybridoma 401-2 (ATCC accession number PTA-5893).

33. Hybridoma 445-1 (ATCC accession number PTA-5894).

34. Hybridoma 445-2 (ATCC accession number PTA-5895).

35. Hybridoma 458-1 (ATCC accession number PTA-5896).

36. A pharmaceutical composition comprising a monoclonal antibody of any one of claims 1, 8, 11 or 14, and a pharmaceutically acceptable carrier.

37. A pharmaceutical composition of claim 36, wherein the monoclonal antibody is humanized.

38. A method of diagnosing a gastrin-mediated disease or condition in a patient, comprising determining the level of a gastrin hormone form in a sample of a biological fluid from the patient and comparing the level of a gastrin hormone form in the sample with the normal level of the gastrin hormone form in a sample of biological fluid from a group of healthy individuals.

39. A method of prevention or treatment of a gastrin-mediated disease or condition comprising administering a pharmaceutical composition comprising a monoclonal antibody of any one of claims 1, 9, 13 or 17 to a patient in need thereof.

40. A method of monitoring the course of a gastrin-mediated disease or condition in a patient, comprising determining the level of a gastrin hormone form in a sample of a biological fluid from a patient suffering from or at risk of a gastrin-mediated disease or condition at a first time point; determining the level of the gastrin hormone form in one or more samples of the biological fluid from the patient at different time points; and thereby monitoring the course of the gastrin-mediated disease or condition.

41. A kit for performing an immunoassay comprising a monoclonal antibody having the characteristics of the monoclonal antibody produced by the hybridoma selected from the group consisting of 400-1, 400-2, 400-3, 400-4, 401-2, 445-1, 445-2, 458-1, and a suitable container.

42. A method of evaluating a gastrin hormone-blocking treatment of a patient suffering from a gastrin hormone-mediated disease or condition, comprising the steps of:
a) obtaining a first sample of biological fluid from the patient prior to or in the early stages of the treatment;
b) determining the level of gastrin hormone in the first sample by an immunoassay method;
c) performing a diagnosis on the basis of the disease or condition to be treated and the level of gastrin hormone in the first sample;
d) administering the treatment to the patient, comprising: a first agent or a substance that generates a first agent which binds gastrin hormone so as to modulate its binding to its target receptor in vivo;
e) obtaining a second sample of biological fluid from the patient after a suitable time within which the treatment would have an effect;
f) determining the level of total gastrin hormone including bound and free gastrin hormone in a first aliquot of the second sample by an immunassay, wherein the first aliquot of the second sample is incubated with (i) a second agent that displaces any gastrin hormone bound by the first agent, and (ii) an immobilized anti-gastrin hormone antibody, wherein the immobilized antibody does not bind the second agent; washing to remove the second agent and adding a detectable antibody that binds the gastrin hormone and does not compete with the immobilized antibody, forming an immunocomplex comprising the immobilized antibody bound to gastrin hormone, the gastrin hormone being bound by the detectable antibody;
g) detecting the amount of the detectable antibody in the immunocomplex and thereby determining the amount of total gastrin hormone in the second sample;
h) determining the level of free gastrin hormone by repeating steps f) and g) with a second aliquot of the second sample, wherein the incubation in step f) is performed without the second agent; and
j) comparing the determined amounts of free gastrin hormone in the first sample with the amounts of free and total gastrin hormone in the second sample so as to determine the efficacy of the gastrin hormone-blocking treatment in the patient.

43. The method of claim 42, wherein the biological fluid is serum.

44. The method of claim 42, wherein the first agent is an antibody to the N-terminus of G17, or a G17 receptor mimic, and the second agent is an N-terminal G17 peptide.

45. The method of claim 44, the substance that generates the first agent is a conjugate comprising a first N-terminal G17 peptide.

46. The method of claim 42, wherein the immobilized antibody binds the C-terminus of G17.

47. The method of claim 46, wherein the immobilized antibody that binds the C-terminus of G17 is a monoclonal antibody.

48. The method of claim 47, wherein the monoclonal antibody has the characteristics of the monoclonal antibody produced by the hybridoma 458-1 (ATCC accession # PTA-5 896).

49. The method of claim 48, wherein the monoclonal antibody is the monoclonal antibody produced by the hybridoma 458-1 (ATCC accession number PTA-5896).

50. The method of claim 44, wherein the detectable antibody binds the N-terminus of G17.

51. The method of claim 50, wherein the antibody selective for the N-terminal of G17 is a monoclonal antibody.

52. The method of claim 51, wherein the monoclonal antibody has the characteristics of the monoclonal antibody produced by the hybridoma selected from the group consisting of hybridoma 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

53. The method of claim 52, wherein the monoclonal antibody is the monoclonal antibody produced by the hybridoma selected from the group consisting of hybridoma 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

54. The method of claim 50, wherein the first agent is an antibody to the N-terminal of G34, and the second agent is an N-terminal G34 peptide.

55. The method of claim 54, wherein the substance that generates the first agent is a conjugate comprising a first N-terminal G34 peptide.

56. The method of claim 54, wherein the immobilized antibody binds the C-terminus of G34.

57. The method of claim 56, wherein the immobilized antibody that binds the C-terminus of G34 is a monoclonal antibody.

58. The method of claim 57, wherein the monoclonal antibody has the characteristics of the monoclonal antibody produced by the hybridoma 458-1 (ATCC accession number PTA-5896).

59. The method of claim 58, wherein the monoclonal antibody is the monoclonal antibody produced by the hybridoma 458-1 (ATCC accession number PTA-5896).

60. The method of claim 54, wherein the detectable antibody binds the N-terminus of G34.

61. The method of claim 60, wherein the antibody that binds the N-terminus of G34 is a monoclonal antibody.

62. The method of claim 61, wherein the monoclonal antibody has the characteristics of the monoclonal antibody produced by the hybridoma selected from the group consisting of hybridoma 401-2 (ATCC accession number PTA-5893).

63. The method of claim 62, wherein the monoclonal antibody is the monoclonal antibody produced by the hybridoma 401-2 (ATCC accession number PTA-5893).

64. The method of claim 42, wherein the first agent is an antibody to the N-terminal of G17-Gly, or a G17-Gly receptor mimic, and the second agent is an N-terminal G17 peptide.

65. The method of claim 64, wherein the substance that generates the first agent is a conjugate comprising a first N-terminal G17 peptide.

66. The method of claim 64, wherein the immobilized antibody binds the C-terminus of G17.

67. The method of claim 60, wherein the immobilized antibody that binds the C-terminus of G17 is a monoclonal antibody.

68. The method of claim 67, wherein the monoclonal antibody has the characteristics of the monoclonal antibody produced by the hybridoma selected from the group consisting of the hybridoma 445-1 (ATCC accession number PTA-5894), and the hybridoma 445-2 (ATCC accession number PTA-5 895).

69. The method of claim 68, wherein the monoclonal antibody is the monoclonal antibody produced by the hybridoma selected from the group consisting of the hybridoma 445-1 (ATCC accession number PTA-5894) and the hybridoma 445-2 (ATCC accession number PTA-5895).

70. The method of claim 64, wherein the detectable antibody binds the N-terminus of G17-Gly.

71. The method of claim 70, wherein the antibody that binds the N-terminus of G17-Gly is a monoclonal antibody.

72. The method of claim 71, wherein the monoclonal antibody has the characteristics of the monoclonal antibody produced by the hybridoma selected from the group consisting of hybridoma 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

73. The method of claim 72, wherein the monoclonal antibody is the monoclonal antibody produced by the hybridoma selected from the group consisting of hybridoma 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

74. The method of claim 42, wherein the first agent is an antibody to the C-terminal of G34-Gly, and the second agent is an N-terminal G34 peptide.

75. The method of claim 74, wherein the substance that generates the first agent is a conjugate comprising a first N-terminal G34 peptide.

76. The method of claim 74, wherein the immobilized antibody binds the C-terminus of G34.

77. The method of claim 76, wherein the immobilized antibody that binds the C-terminus of G34 is a monoclonal antibody.

78. The method of claim 77, wherein the monoclonal antibody has the characteristics of the monoclonal antibody produced by the hybridoma 445-1 (ATCC accession number PTA-5894) and the hybridoma 445-2 (ATCC accession number PTA-5895).

79. The method of claim 78, wherein the monoclonal antibody is the monoclonal antibody produced by the hybridoma 445-1 (ATCC accession number PTA-5894) and the hybridoma 445-2 (ATCC accession number PTA-5895).

80. The method of claim 74, wherein the detectable antibody binds the N-terminus of G34-Gly.

81. The method of claim 80, wherein the antibody that binds the N-terminus of G34-Gly is a monoclonal antibody.

82. The method of claim 81, wherein the monoclonal antibody has the characteristics of the monoclonal antibody produced by the hybridoma selected from the group consisting of hybridoma 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).

83. The method of claim 82, wherein the monoclonal antibody is the monoclonal antibody produced by the hybridoma selected from the group consisting of hybridoma 400-1 (ATCC accession number PTA-5889), hybridoma 400-2 (ATCC accession number PTA-5890), hybridoma 400-3 (ATCC accession number PTA-5891) and hybridoma 400-4 (ATCC accession number PTA-5892).
